(19) United States
(12) Patent Application Publication
(10) Pub. No.: US 2009/0098631 A1
(43) Pub. Date: Apr. 16, 2009

(54) Novel glutamic acid decarboxylase (GAD) proteins and methods of use

(75) Inventors: Helen Lisa Fitzsimons, Palmerston North (NZ); Ross James Bland, Palmerston (NZ)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: NEUROLOGIX, INC., Fort Lee, NJ (US)

(21) Appl. No.: 11/888,556

(22) Filed: Aug. 1, 2007

Related U.S. Application Data
(60) Provisional application No. 60/834,994, filed on Aug. 1, 2006.

(51) Int. Cl. C12N 9/88; C07H 21/00; C12N 5/10; A61K 38/51; A61K 31/7088; C07K 16/40; A61K 9/127; C12N 15/63

(52) U.S. Cl. 435/232; 536/23.2; 435/325; 424/94.5; 514/44; 530/389.1; 530/387.9; 424/450; 435/320.1

(57) ABSTRACT

The invention relates to novel Glutamic Acid Decarboxylases (GAD). More specifically, novel DNA and protein sequences relating to GAD. Additionally, the invention discloses a novel composition and related methods for treating neurodegenerative diseases such as Parkinson's disease, Alzheimer's disease, epilepsy, and the like, using viral and non-viral delivery systems that deliver therapeutic agents to specific regions of the brain. More specifically, using an adeno-associated viral vector to deliver a nucleotide sequence encoding a novel glutamic acid decarboxylase (GAD) to specific regions of the brain that are over stimulated or disinhibited in various diseases, including neurodegenerative diseases.

### CROSS REFERENCE TO RELATED APPLICATIONS

This application claims priority to U.S. Provisional Patent Application No. 60/834,994 filed on Aug. 1, 2006 and entitled “Novel Glutamic Acid Decarboxylase (GAD) Proteins And Methods Of Use” which is hereby incorporated by reference in its entirety.

### FIELD OF THE INVENTION

The invention relates to novel Glutamic Acid Decarboxylases (GAD). More specifically, the invention contemplates novel GAD DNA and protein sequences. Additionally, the invention discloses a novel composition and related methods for treating neurodegenerative diseases such as Parkinson's disease, Alzheimer's disease, epilepsy, and the like, using viral and non-viral delivery systems that deliver therapeutic agents to specific regions of the brain. More specifically, the invention contemplates using an adeno-associated viral vector to deliver a nucleotide sequence encoding a novel glutamic acid decarboxylase (GAD) to specific regions of the brain that are over stimulated or disinhibited in various diseases, including neurodegenerative diseases.

### BACKGROUND OF THE INVENTION

### I. Glutamic Acid Decarboxylase (GAD) and Gamma-aminobutyric Acid (GABA)

Gamma aminobutyric acid (GABA) and glutamic acid are two major neurotransmitters involved in the regulation of brain neuronal activity. GABA is the major inhibitory neurotransmitter and L-glutamic acid is an excitatory transmitter (Roberts et al., GABA IN NERVOUS SYSTEM FUNCTION, Raven Press: New York, 1976; McGeer et al., GLUTAMINE, GLUTAMATE, AND GABA IN THE CENTRAL NERVOUS SYSTEM; Hertz L, Kvamme E, McGeer E G, Schousbal A, eds., Liss: New York, 1983; 3-17). Loss of GABA signaling, by a reduction in release, loss of neurons which synthesize GABA, or antagonism of GABA receptors leads to disinhibition, overexcitation and depending on the specific brain region involved, may result in epilepsy, movement disorders or other neurological deficits and symptoms.

GABA is released from dopaminergic cells. An imbalance in the concentration of these neurotransmitters can lead to convulsive states. When the concentration of GABA diminishes below a threshold level in the brain, convulsions result (Karlsson et al., (1974) Biochem. Pharmacol. 23:3053-3061). When the GABA levels rise in the brain the convulsions terminate (Hayashi, T. (J. Physiol. 145(3) 570-8 (1959) supra). In several convulsive disorders there is concomitant with reduced brain GABA levels a diminished level of glutamic acid decarboxylase (GAD) activity (McGeer et al., GABA IN NERVOUS SYSTEM FUNCTION; Roberts E, Chase T N, Tower D B, eds., Raven Press: New York 1976:487-495; Butterworth et al., (1983) Neurochem. 41:440-447). The concentrations of GAD and GABA vary in parallel because decreased GAD concentration results in lower GABA production.

GABA interacts with a least two receptors, GABA-A and GABA-B. GABA-A receptors have been well characterized and are coupled to chloride channels (Bormann (1988) Trends Neurosci. No. 11: 112-116). GABA-A receptors are related to ligand gated ion channels belonging to the same superfamily as the nicotrinic receptor for achetylcholine. In contrast, GABA-B receptors are less well understood, although reports describe that the GABA-B receptors are coupled to either calcium or potassium channels (Bormann (1988), supra).

The majority of neurons in the striatum (caudate-putamen, dorsal striatum; nucleus accumbens, ventral striatum) and in striatal projection regions (the pallidum, the entopeduncular nucleus and substantia nigra reticulata) use GABA as transmitter and express GAD in the synthesis of GABA.

There are two main forms of GAD present in the vertebrate brain, GAD65 and GAD67, which are the products of two separate genes Proc Natl Acad Sci 89:2115-2119. (Bu et al., 1992). Both forms of the protein are co-expressed throughout the brain but differ in their structure, subcellular localization and regulation. These differences suggest the two GAD isoforms may play differing roles in GABA-mediated neurotransmission.

Human GAD65 and GAD67 have been previously isolated and cloned by Bu et al. (1992). Human GAD65 cDNA encodes a Mr 65,000 polypeptide, with 585 amino acid residues (Genbank Accession No. NM000818;M81882), (SEQ ID No: 11) Human GAD67 encodes a Mr 67,000 polypeptide, with 594 amino acid residues (Genbank Accession No. NM013445;M81883).

Human GAD proteins are comprised of two distinct domains. The C-terminal domain, which contains the catalytic site and cofactor binding site, is relatively conserved between human GAD65 and GAD67 with 73% identity. The N-terminus, which contains a membrane association domain, is highly divergent with only 23% identity (Bu et al., 1992).

Targeting of GAD65 to the Golgi is mediated by a 27 amino acid domain in the N-terminus, which is not present in GAD67. In CHO and COS cells, membrane association of GAD67 is dependent on the presence of GAD65, presumably through heterodimer formation (Dirkx R, et al., 1995, J. Biol. Chem., 270(5): 2241-6). Targeting to presynaptic clusters is mediated by a palmitoylated 60 amino acid N-terminal domain of GAD65 (Kanaani et al., (J. Cell. Biol. 158(7): 1229-38 (2002).

An immunoprecipitation study determined that 33% of GAD protein in rat brain extract is present as GAD65/67 heterodimers (Kanaani et al., J. Biol. Chem., 274(52) 37-200-9 (1999). Similarly, in another study 27% of GAD protein isolated from rat cerebellum was in the form of GAD65/67 heterodimers (Sheikh and Martin, J. Neurocher 66(5):2082-90 (1996). GAD67 has, however, been found to associate with membranes in GAD65−/− mice, suggesting that axonal targeting and membrane association can occur via a mechanism independent of GAD65 (Kanaani et al., 1999).

Both GAD65 and GAD67 require the presence of the cofactor pyridoxal phosphate (PLP) for enzyme activity (Martin et al., 1991). Half of the GAD65 protein occurs in the inactive apoenzyme form without bound PLP whereas GAD67 occurs mostly in the active holoenzyme form (Erlander et al., 1991). This inactive pool of “stored” GAD has been postulated to be available for activation at times of high or sudden demand for GABA.

There are marked differences in the amount and activity of GAD protein in different areas of the rat brain. The amount of GAD65 was found by immunoblotting to be 77-89% of total GAD protein in twelve brain areas analysed which correlated with total GAD activity (Sheikh et al., 355(1): 73-80 (1999),

Although GAD65 is the predominant form of GAD present in rat brain, there is evidence from knockout mouse studies that GAD67 synthesises the majority of GABA in the brain. GAD67^{-/-} mice do not display defects in brain morphology at birth but die soon after due to a cleft palate (Asada et al., 1997; Condie et al., 1997). GAD activity and GABA content in the cerebral cortex is reduced to 20% and 7% respectively in newborn GAD67^{-/-} mice (Asada et al., 1997). GAD65−/− mice are viable but GABA levels are low for the first two months after birth (Stork et al., 2000). Adult rats with this mutation display abnormal neural activity with spontaneous seizures and paroxysmal discharges (Kash et al., 1997). They also have increased susceptibility to picrotoxin induced seizures than their wild type litter mates (Asada et al., 1996). From these observations, it is obvious that although GAD65 and GAD67 contribute to a metabolic pool of GABA, their roles with respect to inhibitory neurotransmission are different. It is possible that due to its presence throughout the neurons, predominantly in the holoenzyme form, GAD67 may contribute to the basic requirements of inhibitory neurotransmission. The low saturation of GAD65 by PLP, combined with the subcellular distribution in axon terminals and anchoring to synaptic vesicles suggest that GAD65 may be involved in the prevention of hyperexcitability by its rapid activation and loading of GABA into vesicles for rapid secretion.

### II. Neurological and Other Disorders

Diseases such as Parkinson's disease, Huntington's disease, Amyotrophic Lateral Sclerosis (ALS or Lou Gehrig's Disease), Epilepsy and Alzheimer's disease, have proved difficult to treat. Few, if any therapies, have proved effective in slowing or arresting the degenerative process associated with these diseases.

In Parkinson's Disease (PD), the primary neurochemical disturbance is believed to be the loss of substantia nigra (SN) dopaminergic (DA) neurons. This loss of DA neurons leads to a profound deficit of DA in the projection areas of the caudate and putamen and results in a loss of signaling through dopamine receptors in the postsynaptic neurons. These neurons, via efferents referred to as the direct and indirect pathways, synapse on other cells in the basal ganglia circuitry. Of most relevance to PD, the loss of dopamine receptors in the basal ganglia circuitry leads to loss of drive in the GABAergic inhibitory input to the subthalamic nucleus.

The loss of inhibitory GABAergic drive to the subthalamic nucleus (STN) results in increased activity of the STN which sends excitatory (glutamatergic) afferents to the ventrial media (VM) thalamus, the substantia nigra pars reticulata (SNPR) and a lesser projection to the pars compacta, as well as other cells within the basal ganglia including the globus pallidus. When the concentration of GABA diminishes below a threshold level in the brain, movement disorders and convulsions may result (See e.g. Karlsson et al, (1974) Biochem. Pharmacol 23:3053-3061). GABA synthesis is regulated by glutamic acid decarboxylase (GAD). GAD is present in the brain as two isoforms, GAD65 and GAD67. When the GABA levels rise in the brain the convulsions terminate (See e.g., Hayashi (1959). In convulsive disorders, the reduction in brain GABA levels is often paralleled by a diminished level of GAD (McGeer, et al. GABA IN NERVOUS SYSTEM FUNCTION; Roberts E, Chase T N, Tower D B, eds., Raven Press: New York 1976:487-495; Butterworth et al. (1983) Neurochem. 41:440-447; Spokes et al. (1978) Adv. Exp. Med. Biol. 123:461-473).

Levodopa (L-dopa) has historically been the medication of choice to treat Parkinson's disease. L-dopa is a precursor to dopamine and is able to cross the blood-brain barrier to target the brain. Unfortunately, the response with L-dopa is not sustainable. Most patients develop adverse effects after long-term usage of L-dopa, and often the benefits of treatment wane as the disease progresses.

Other methods for treating Parkinson's disease include transplantation of cells used to repair regions of the brain damaged by neurodegeneration. These cells can be engineered to secrete neuroactive substances such as L-dopa. The procedure typically involves cell transplantation into the stratium. However, cell transplantation is a complicated procedure which requires donor tissue, and there have been reports of mortality associated with this procedure.

Alternative forms of treating Parkinson's disease involve implanting devices for deep-brain stimulation (DBS) in specific regions of the brain. For example, DBS of the STN. These devices are typically electrodes implanted into the STN. The electrode is then stimulated at a desired frequency to reduce the effect of Parkinson's disease. The significance of the STN overactivity is reflected in the success of ablative surgery of the STN in both animal models of Parkinson's disease, as well as in human Parkinson's disease itself. In addition to ablation, implantation of electronic stimulators are commonly employed. The mechanism of the stimulators is believed to be mediated by local inhibition (via GABA signaling), and is replicated by the local infusion of GABA agonists.

Like Parkinson's disease, methods for treating epilepsy include the use of anti-epileptic drugs, such as sodium valporate (Epilim). Available drugs reduce seizure frequency in the majority of patients, but it is estimated that only about forty percent are free of seizures despite optimal treatment. Other forms of treatment include DBS of certain regions of the brain, such as the VIM (ventral intermediate thalamus), subthalamic nucleus, and internal globus pallidus. However, the DBS procedure is not always effective in many patients who require repeated treatment.

Each of these approaches, surgical ablation, electrical stimulation and infusion of pharmacological GABA agonists is effective in disease palliation, but each has significant adverse effects. For example, extensive invasive surgery, a high risk of infection and potential damage to the brain and in the case of drug infusion, very transient efficiency.

Thus, the treatments for neurodegenerative disorders are palliative at best, with limited and transient efficacy. Therefore, a need exists for a therapeutic approach which has advantages in targeting specificity, both short and long-term efficacy, as well as neuroprotection, without extensive surgery or side-effects.

### SUMMARY OF THE INVENTION

The invention is drawn to novel glutamic acid decarboxylases (GAD). More specifically this invention relates to novel GAD polypeptides and polynucleotides which encode novel GAD polypeptides (“Novel GAD”), antibodies and other molecules which bind the polypeptides and/or polynucleotides of the invention, compositions comprising the polypeptides, polynucleotides or antibodies or other binding molecules of the invention as well as methods of use of any of the foregoing.

More specifically, one aspect of the invention provides for novel feline GAD (“Feline GAD”) polypeptides. The invention also provides polypeptides that have substantial homology to the foregoing Feline GAD polypeptides, modified forms of the novel Feline GAD polypeptides fragments of the polypeptides. The invention also includes successors or metabolites of the novel chimeric polypeptides in biological pathways. The invention also provides molecules that comprise a novel chimeric polypeptide, homologous polypeptide, a modified novel chimeric polypeptide or a fragment, successor or metabolites polypeptide marker (e.g., a fusion proteins).

An additional aspect of the invention provides for novel consensus GAD polypeptides. The invention also provides polypeptides that have substantial homology to the foregoing novel consensus GAD polypeptides, modified forms of the novel Consensus GAD polypeptides fragments of the polypeptides. The invention also includes successors or metabolites of the novel chimeric polypeptides in biological pathways. The invention also provides molecules that comprise a novel chimeric polypeptide, homologous polypeptide, a modified novel chimeric polypeptide or a fragment, successor or metabolites polypeptide marker (e.g., a fusion proteins). As used herein, the term “polypeptides of the invention” shall be understood to include all of the foregoing.

Another aspect of the invention provides polynucleotides encoding polypeptides of the invention (“novel polynucleotides”). The invention also provides polynucleotides that have substantial homology to novel polynucleotides, modified novel polynucleotides, and fragments of novel polynucleotides. The invention also provides molecules that comprise a novel polynucleotide, homologous polynucleotide, modified novel polynucleotides or a fragments of novel polynucleotides (e.g., a vector). The novel polynucleotides of the present invention are intended to include analogs, compounds having a native polypeptide sequence and structure with one or more amino acid additions, substitutions (generally conservative in nature) and/or deletions, relative to the molecule of the invention, so long as the modifications do not alter the differential expression of the marker. As used herein, the term “polynucleotides of the invention” shall be understood to include all of the foregoing.

Another aspect of the invention provides molecules that specifically bind to a polypeptide of the invention, metabolite of the invention or polynucleotide of the invention. The binding molecule may be an antibody, antibody fragment, or other molecule. The invention also provides methods for producing a binding molecule that specifically recognizes a polypeptide of the invention, metabolite of the invention or polynucleotide of the invention.

Another aspect of the invention provides compositions comprising a polypeptide of the invention, metabolite of the invention or polynucleotide of the invention, a binding molecule (e.g., an antibody) that is specific for a polypeptide of the invention, metabolite of the invention or polypeptide of the invention, an inhibitor of a polypeptide of the invention, metabolite of the invention or polynucleotide of the invention, or another molecule that can increase or decrease the level or activity of a polypeptide of the invention, metabolite of the invention or polynucleotide of the invention. Such compositions may be pharmaceutical compositions formulated for use as therapeutics.

Another aspect of the invention provides methods for treating neurodegenerative disorders (such as Parkinson's Disease or Alzheimer's Disease) by administering a therapeutic agent to a subject that increases or decreases the level or activity of a polypeptide of the invention, metabolite of the invention or polynucleotide of the invention. For polypeptides of the invention, metabolites of the invention or polynucleotides of the invention that are increased in samples obtained from a subject with a neurodegenerative disorder, the method comprises administering a therapeutic agent that decreases (i.e., bring toward the normal range) the level or activity of the polypeptide, metabolite or polynucleotide. Similarly, for polypeptides of the invention, metabolites of the invention or polynucleotides of the invention that are decreased in samples obtained from a subject with a neurodegenerative disorder, the method comprising administering a therapeutic agent that increases the level or activity of the polypeptide, metabolite or polynucleotide.

Another aspect of the invention provides a method for detecting a polypeptide of the invention, metabolite of the invention or polynucleotide of the invention. In one embodiment, the method comprises contacting a biological sample obtained from a subject with a binding molecule (e.g., an antibody) under conditions that permit the formation of a stable complex, and detecting any stable complexes formed. In another embodiment, the method comprises determining the activity of a polypeptide of the invention, metabolite of the invention or polynucleotide of the invention. In another embodiment, the method comprises determining the level of a polypeptide of the invention in a cell obtained from the subject by detecting the presence of a polynucleotide that encodes the polypeptide.

Another aspect of the invention provides compositions comprising a polypeptide of the invention, metabolite of the invention or polynucleotide of the invention, a binding molecule (e.g., an antibody) that is specific for a polypeptide of the invention, metabolite of the invention or polypeptide of the invention, an inhibitor of a polypeptide of the invention, metabolite of the invention or polynucleotide of the invention, or another molecule that can increase or decrease the level or activity of a polypeptide of the invention, metabolite of the invention or polynucleotide of the invention. Such compositions may be pharmaceutical compositions formulated for use as therapeutics.

The invention is also based, at least in part, on the discovery that localized delivery of a vector comprising a therapeutic agent to a specific region of the brain that is overstimulated or disinhibited in neurodegenerative diseases, can reduce the effect of overstimulation and promote the improvement of the neurodegenerative disease. In particular, the invention pertains to methods and compositions used to deliver a vector, (e.g., an adeno-associated virus vector (AAV)) comprising a nucleotide sequence encoding a consensus glutamic acid decarboxylase (Consensus GAD65) to target relevant cells, such as the hippocampus or the subthalamic nucleus of the basal ganglia, Particularly preferred methods of delivering the vector to specific regions of the brain are those techniques that are simple, safe, and have a lower risk associated with them than lesioning, electrode implantation or cell transplantation. For example, delivery of the vector using stereotactic microinjection techniques, or delivery of the vector using specialized probes, or percutaneous delivery via disruption of the blood-brain barrier. Delivery of the vector using the method of the invention results in minimal immunological or inflammatory responses within the regions of the brain, thus eliminating the need for immunosupression. After delivery of the vector to a specific region of the brain, regional dispersion and/or diffusion of vector occurs ensuring local distribution of gene and stable gene expression.

The methods and compositions are particularly useful for treating neurodegenerative diseases, such as Parkinson's disease, Huntington's disease, Amyotrophic Lateral Sclerosis (ALS or Lou Gehrig's Disease), Alzheimer's Disease as well as epilepsy.

Accordingly, the invention is directed to a method for treating a neurodegenerative disease in a subject identifying a target site in the central nervous system that requires modification. A vector comprising a nucleotide sequence encoding a Feline GAD65 or Consensus GAD65 is then delivered to the target site in the central nervous system. The Feline GAD65 or Consensus GAD65 is expressed in the target site to treat or reduce the neurodegenerative disease.

In one embodiment, the vector is a viral vector, and is selected from the group consisting of adenovirus vectors, herpes virus vectors, parvovirus vectors, and lentivirus vectors. In a preferred embodiment, the viral vector is an adeno-associated viral vector.

In another embodiment, the vector is a non-viral vector. In a preferred embodiment, the non-viral vector is a liposome-mediated delivery vector.

In one embodiment, the vector is delivered to a specific target site of the central nervous system. In a preferred embodiment, the vector is delivered using stereotaxic delivery, or delivery using specialized probes. In a preferred embodiment, the target site of the central nervous system is a region of the brain. In another preferred embodiment, the region of the brain is selected from the group consisting of basal ganglia, subthalamic nucleus (STN), pedunculopontine nucleus (PPN), substantia nigra (SN), thalamus, hippocampus, amygdala, hypothalamus, cortex and combinations thereof. In one embodiment, the neurodegenerative disease is selected from the group consisting of Parkinson's disease and related movement disorders, Alzheimer's disease, senile dementia, Amyloid Lateral Schlerosis (ALS), chronic pain and epilepsy.

In another aspect, the invention pertains to a method for treating epilepsy in a subject by identifying one or more regions of the brain that require modification. An adeno-associated viral (AAV) vector comprising a nucleotide sequence encoding a Feline GAD65 is delivered to the region of the brain, and GAD in expressed the region of the brain to treat or reduce epilepsy.

In another aspect, the invention pertains to a method for treating Parkinson's Disease in a subject by identifying one or more regions of the brain that require modification. An adeno-associated viral (AAV) vector comprising a nucleotide sequence encoding a Feline GAD65 is delivered to the region of the brain, and GAD in expressed the region of the brain to treat or reduce epilepsy.

In another aspect, the invention pertains to a method for treating epilepsy in a subject by identifying one or more regions of the brain that require modification. An adeno-associated viral (AAV) vector comprising a nucleotide sequence encoding a Consensus GAD65 is delivered to the region of the brain, and GAD in expressed the region of the brain to treat or reduce epilepsy.

another aspect, the invention pertains to a method for treating Parkinson's Disease in a subject by identifying one or more regions of the brain that require modification. An adeno-associated viral (AAV) vector comprising a nucleotide sequence encoding a Consensus GAD65 is delivered to the region of the brain, and GAD in expressed the region of the brain to treat or reduce epilepsy

In yet another aspect, the invention pertains to a vector for expression of Feline GAD or Consensus GAD in cells of the central nervous system comprising a tissue specific promoter operably linked to a nucleotide sequence encoding Feline or Consensus GAD, and a post-transcriptional regulatory element.

In one embodiment, the promoter is specific for central nervous system cells and tissues, such as the cells and tissues of the brain. In a preferred embodiment, the promoter is the neuron specific enolase (NSE) promoter.

### BRIEF DESCRIPTION OF FIGURES

FIG. 1 is a depiction of a (double stranded) Feline GAD65 DNA sequence (SEQ. ID No. 1)

FIG. 2 is a depiction of the Feline GAD65 amino acid sequence (SEQ. ID No. 2)

FIG. 3A. is a Western Blot of GAD proteins from human (hGAD65, hGAD67) and cat. The GAD proteins were run alongside samples of recombinant human GAD65 (rhGAD65) of known amounts (10 to 80 ng). EGFP (29 kDa) was used as a transfection control.

FIG. 3B. is a depiction of the level of Feline GAD65, human GAD65 and human GAD67. The proteins were quantified after generation of a standard curve from recombinant human GAD65 protein, and normalization to the EGFP loading control.

FIG. 4 shows the expression of Feline GAD65 in the rat subthalamic nucleus. Numerous GAD65 positive cell bodies can be seen on the injected side. Arrows point to some of the cells.

FIG. 5 depicts the methodology for calculating the Consensus GAD polypeptide sequence. GAD65 amino acid sequences were aligned across eight mammalian species in order to generate a consensus sequence, including feline (SEQ ID NO. 2), canine (SEQ ID NO. 5), murine (SEQ ID NO 6), bovine (cow) (SEQ ID NO. 7), Porcine (SEQ ID NO. 8), Macaque (SEQ ID NO. 9), rat (SEQ ID NO. 10) and human (SEQ ID No. 11).

FIG. 6 is a depiction of the (double stranded) Consensus GAD65 DNA sequence (SEQ. ID NO. 3).

FIG. 7 is a depiction of the Consensus GAD65 polypeptide sequence (SEQ. ID No. 4).

FIG. 8 shows the number of amino acid differences between cat, human, rat and consensus GAD65 across amino acids 1 to 100, and from amino acids 101 to 585.

FIG. 9A. is a Western Blot of consensus GAD protein (cons GAD65) compared to human GAD65, human GAD67 and feline GAD65. The GAD proteins were run alongside samples of recombinant human GAD65 (rhGAD65) of known amounts (10 to 60 ng). EGFP (29 kDa) was used as a transfection control.

FIG. 9B. is a depiction of the level of human GAD65, human GAD67, consensus GAD65 and feline GAD65. The proteins were quantified after generation of a standard curve from recombinant human GAD65 protein, and normalization to the EGFP control.

FIG. 10A. is a Western Blot of consensus GAD65 (consGAD65) and human GAD65 (hGAD65), each co-transfected with human GAD67 (hGAD67). The GAD proteins were run alongside samples of recombinant human GAD65 (rhGAD65) of known amounts (10 to 60 ng). EGFP (29 kDa) was used as a loading control.

FIG. 10B. is a depiction of the combined level of GAD protein from consensus GAD65 and human GAD67 compared to that from human GAD65 and human GAD67 combined. The proteins were quantified after generation of a standard curve from recombinant human GAD65 protein, and normalization to the EGFP loading control.

FIGS. 11A-11C. depict an analysis of GABA activity from human GAD67 combined with either human or consensus GAD65. FIG. 10A shows Basal GABA release, FIG. 10B shows Glutamic acid release and FIG. 10C shows the ratio of GABA release to glutamic acid release.

### DETAILED DESCRIPTION

The practice of the present invention employs, unless otherwise indicated, conventional methods of virology, microbiology, molecular biology and recombinant DNA techniques within the skill of the art. Such techniques are explained fully in the literature. (See, e.g., Sambrook, et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, Vol. I & II (P. Tijessen, ed.); Fundamental Virology, 2nd Edition, Vol. I & II (B. N. Fields and D. M. Knipe, eds.)).

### I. Definitions

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered. The term “central nervous system” or “CNS” as used herein refers to the art recognized use of the term. The CNS pertains to the brain, cranial nerves and spinal cord. The CNS also comprises the cerebrospinal fluid, which fills the ventricles of the brain and the central canal of the spinal cord.

The term “modifies” or “modified” are used interchangeably herein and refer to the up-regulation or down-regulation of a target gene or a target protein. The term modifies or modified also refers to the increase, decrease, elevation, or depression of processes or signal transduction cascades involving a target gene or a target protein. For example, a target protein can be a GABA. Modification to the GABA concentrations may occur when a therapeutic agent, e.g., Consensus GAD, or Feline GAD alter GABA concentration. For example, modifications that result in an increase in GABA concentration by the expression of a novel GAD in glutaminergic neurons and intrinsic cells of the STN. Modifications can also result from the addition of a therapeutic agent that inactivates GABA aminotransferase. The effect is to block the degradation of GABA and thereby increase its concentration. Numerous mechanism-based inactivators of GABA aminotransferase are known (See e.g., Silverman Mechanism-Based Enzyme Inactivation: Chemistry and Enzymology, Vol. I and II, CRC: Boca Raton 1988).

Non-limiting examples of modifications includes modifications of morphological and functional processes, under- or over production or expression of a substance or substances, e.g. a neurotransmitter, by neural cells, failure of neural cells to produce a substance or substances which it normally produces, production of substances, e.g., neurotransmitters, and/or transmission of electrical impulses.

The term “tissue-specific promoter” as used herein refers to a promoter that is operable in cells of the central nervous system (CNS). Examples of promoters for the CNS include but are not limited to, neuron-specific promoters (e.g., the neurofilament promoter; Byrne and Ruddle (1989) Proc. Natl. Acad. Sci. USA 86:5473-5477) and glial specific promoters (Morii et al. (1991) Biochem. Biophys Res. Commun. 175: 185-191). Preferably, the promoter is tissue specific and is essentially not active outside the central nervous system, or the activity of the promoter is higher in the central nervous system that in other systems. For example, a promoter specific for the spinal cord, brainstem, (medulla, pons, and midbrain), cerebellum, diencephalon (thalamus, hypothalamus), telencephalon (corpus stratium, cerebral cortex, or within the cortex, the occipital, temporal, parietal or frontal lobes), STN, SN, or combinations, thereof. The promoter may also be one that can be used in combination with an AAV to result in higher expression. For example, a cytomegalovirus enhancer/chicken-Actin (CBA) hybrid promoter that functions in cells of the CNS (Xu et al. (2001) Hum Gene Ther. 12:563-73).

The terms “neurodegenerative disorder” or a “neurological disorder” as used herein refers to a disorder which causes morphological and/or functional abnormality of a neural cell or a population of neural cells. The neurodegenerative disorder can result in an impairment or absence of a normal neurological function or presence of an abnormal neurological function in a subject. For example, neurodegenerative disorders can be the result of disease, injury, and/or aging. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. Neurodegeneration can occur in any area of the brain of a subject and is seen with many disorders including, for example, head trauma, stroke, ALS, multiple sclerosis, Huntington's disease, Parkinson's disease, and Alzheimer's disease.

As used herein, the terms “Parkinson's subject” and “a subject who has Parkinson's” are intended to refer to subjects who have been diagnosed with Parkinson's or probable Parkinson's. The terms “non-Parkinson's subject” and “a subject who does not have Parkinson's” are intended to refer to a subject who has not been diagnosed with Parkinson's or probable Parkinson's. A non-Parkinson's subject may be healthy and have no other disease, or they may have a disease other than Parkinson's. The term “subject,” as used herein, refers to any living organism capable of eliciting an immune response. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, Parkinson's adult and newborn subjects, as well as fetuses, whether male or female, are intended to be covered.

As used herein, the term “antibody” refers to an immunoglobulin molecule capable of binding an epitope present on an antigen. The term is intended to encompasses not only intact immunoglobulin molecules such as monoclonal and polyclonal antibodies, but also bi-specific antibodies, humanized antibodies, chimeric antibodies, anti-idiopathic (anti-ID) antibodies, single-chain antibodies, Fab fragments, F(ab′) fragments, fusion proteins and any modifications of the foregoing that comprise an antigen recognition site of the required specificity.

As used herein, the term “biological sample” includes a sample from any body fluid or tissue (e.g., serum, plasma, blood, cerebrospinal fluid, urine, sputum, thin cortical slice, brain tissue homogenate).

As used herein, a component (e.g., a marker) is referred to as “differentially expressed” in one sample as compared to another sample when the method used for detecting the component provides a different level or activity when applied to the two samples. A component is referred to as “increased” in the first sample if the method for detecting the component indicates that the level or activity of the component is higher in the first sample than in the second sample (or if the component is detectable in the first sample but not in the second sample). Conversely, a component is referred to as “decreased” in the first sample if the method for detecting the component indicates that the level or activity of the component is lower in the first sample than in the second sample (or if the component is detectable in the second sample but not in the first sample). In particular, marker is referred to as “increased” or “decreased” in a sample (or set of samples) obtained from an AD subject (or a subject who is suspected of having AD, or is at risk of developing AD) if the level or activity of the marker is higher or lower, respectively, compared to the level of the marker in a sample (or set of samples) obtained from a non-AD subject, or a reference value or range.

As used herein, the term “polypeptide” refers to a single amino acid or a polymer of amino acid residues. A polypeptide may be composed of two or more polypeptide chains. A polypeptide includes a protein, a peptide, an oligopeptide, and an amino acid. A polypeptide can be linear or branched. A polypeptide can comprise modified amino acid residues, amino acid analogs or non-naturally occurring amino acid residues and can be interrupted by non-amino acid residues. Included within the definition are amino acid polymers that have been modified, whether naturally or by intervention, e.g., formation of a disulfide bond, glycosylation, lipidation, methylation, acetylation, phosphorylation, or by manipulation, such as conjugation with a labeling component.

As used herein, a “fragment” of a polypeptide refers to a plurality of amino acid residues comprising an amino acid sequence that has at least 5 contiguous amino acid residues, at least 10 contiguous amino acid residues, at least 20 contiguous amino acid residues or at least 30 contiguous amino acid residues of a sequence of the polypeptide. As used herein, a “fragment” of polynucleotide refers to a single nucleic acid or to a polymer of nucleic acid residues comprising a nucleic acid sequence that has at least 15 contiguous nucleic acid residues, at least 30 contiguous nucleic acid residues, at least 60 contiguous nucleic acid residues, or at least 90% of a sequence of the polynucleotide. In the present invention, the terms “fragments,” “analogs,” or “derivatives” are used interchangeably to mean a chemical substance that is related to another substance (i.e., marker). The fragment can be, for example, differentially expressed in one sample compared to another sample. In a preferred embodiment, the fragment is differentially expressed in samples from AD subjects when compared to non-AD subjects.

As used herein, a compound is referred to as “isolated” when it has been separated from at least one component with which it is naturally associated. For example, a polypeptide can be considered isolated if it is separated from contaminants including metabolites, polynucleotides and other polypeptides. Isolated molecules can be either prepared synthetically or purified from their natural environment. Standard quantification methodologies known in the art can be employed to obtain and isolate the molecules of the invention.

As used herein, the term “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

In some embodiments, a polypeptide is a member of a biological pathway. As used herein, the term “precursor” or “successor” refers to molecules that precede or follow the polypeptide. Thus, once a polypeptide is identified as a member of one or more biological pathways, the present invention can include additional members of the biological pathway that come before or follow the polypeptide. Such identification of biological pathways and their members is within the skill of one in the art.

As used herein, the term “specifically binding,” refers to the interaction between binding pairs (e.g., an antibody and an antigen) with an affinity constant of at most 10⁻⁶ moles/liter, at most 10⁻⁷ moles/liter, or at most 10⁻⁸ moles/liter.

As used herein, two polypeptides are “substantially homologous” when there is at least 70% homology, at least 80% homology, at least 90% homology, at least 95% homology or at least 99% homology between their amino acid sequences, or when polynucleotides encoding the polypeptides are capable of forming a stable duplex with each other. Likewise, two polynucleotides are “substantially homologous” when there is at least 70% homology, at least 80% homology, at least 90% homology, at least 95% homology or at least 99% homology between their amino acid sequences or when the polynucleotides are capable of forming a stable duplex with each other. In general, “homology” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis of similarity and identity, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence similarity and identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent similarity of a particular nucleotide sequence to a reference sequence can be determined using the homology algorithm of Smith and Waterman with a default scoring table and a gap penalty of six nucleotide positions. Alternatively, homology can be determined by hybridization of polynucleotides under conditions that form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

The phrase “first phenotypic state” and “second phenotypic state” refers to a cell that displays a particular characteristic typical for that cell. The characteristics can be modified or altered, by genetic intervention, into a second phenotypic state such that the cell displays a characteristic that is different from the original characteristic. For example, delivering GAD to glutamatergic excitatory neurons changes their phenotypic characteristic from excitatory neurons to inhibitory neurons

The invention is described in more detail in the following subsections:

### II. Feline GAD65

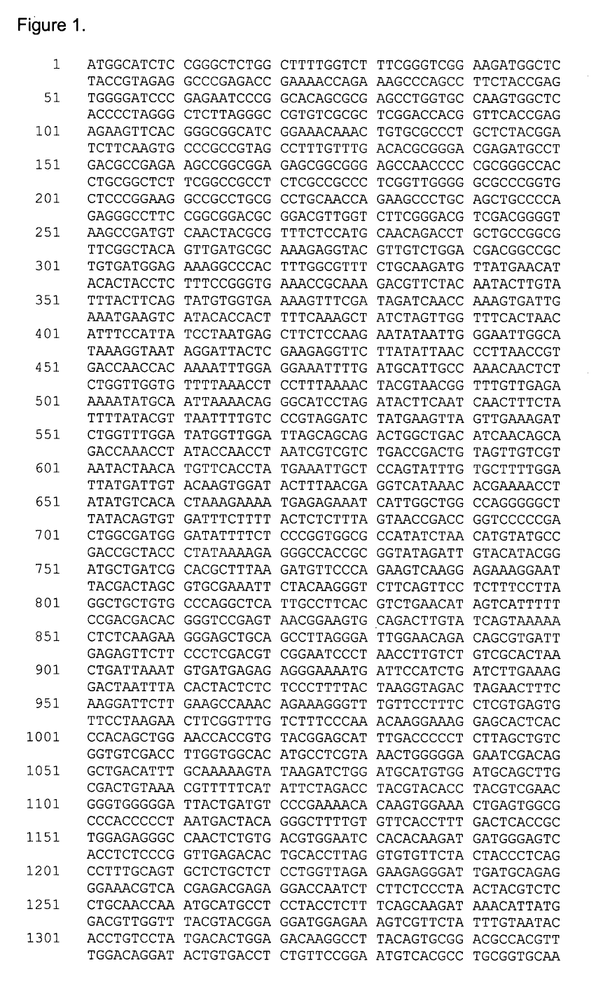

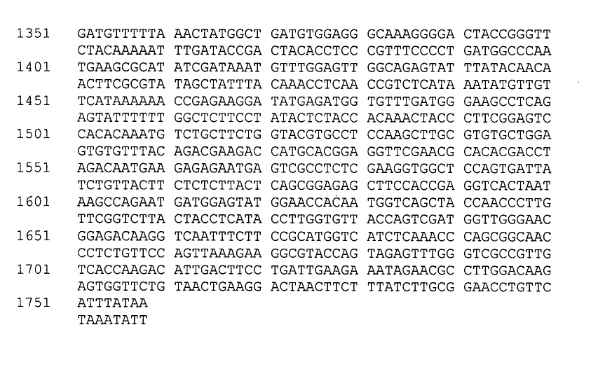

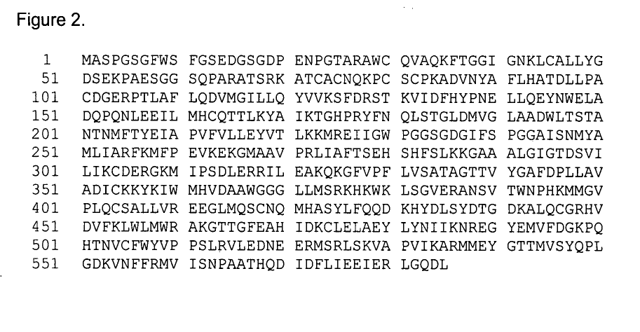

The invention comprises, in part, certain novel GAD polynucleotide and polypeptide sequences. In one aspect of the invention, these novel sequences comprise a novel GAD65 isolated from feline sources (“Feline GAD65”) (FIG. 1, SEQ. ID No. 1 and FIG. 2 SEQ. ID No. 2).

In another embodiment, the invention comprises a polynucleotide sequence coding for a Feline GAD65 polypeptide. Preferably, the invention comprises a polynucleotide sequence coding for polypeptide sequence comprised of the polypeptide of SEQ ID NO. 2. More preferably the polynucleotide coding for the Feline GAD65 polypeptide is the polynucleotide of SEQ. ID No. 1.

The cat GAD65 open reading frame was cloned from a cat brain cDNA. The DNA sequence is shown in FIG. 1 and the amino acid sequence is shown in FIG. 2. There are sixteen amino acid differences between human and cat GAD65.

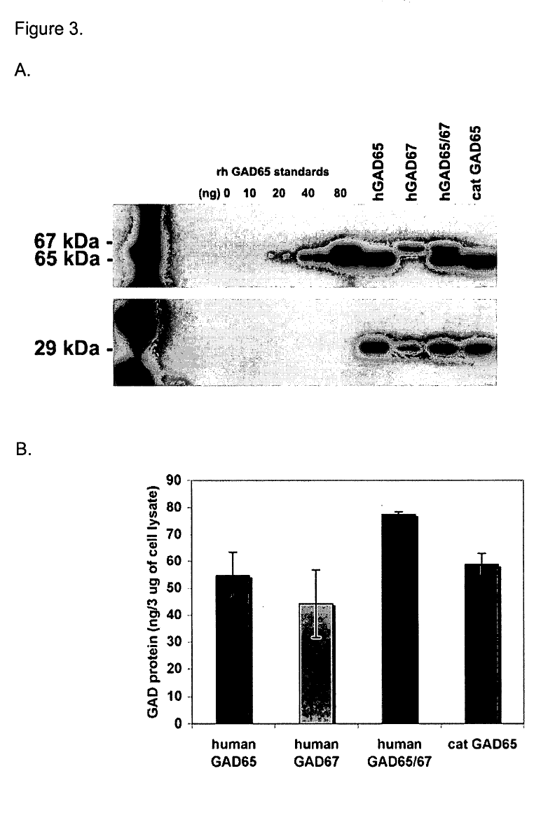

Expression of cat GAD65 protein was analyzed in vitro by transfection of HEK293 cells with plasmids expressing human GAD65, human GAD67 and cat GAD65. Western blots were probed with an antiGAD65/GAD67 antibody show that the cat GAD65 protein is the same size as human GAD65 (FIG. 3A) and level of protein produced from each isoform is equal (FIG. 3B).

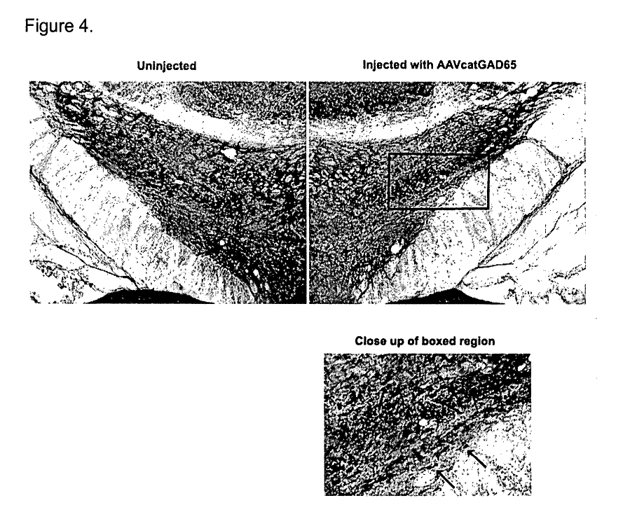

In order to analyze in vivo expression of cat GAD65, recombinant adeno-associated virus vectors were packaged and injected into the rat subthalamic nucleus. Cat GAD65 expression was analyzed by immunohistochemistry four weeks post-injection (FIG. 4). An increase in GAD65 stained cells can be seen on the injected side

### III. Consensus GAD

The invention also comprises polypeptide and polynucleotide sequences which code for an artificial glutamic acid decarboxylase (“Consensus GAD”).

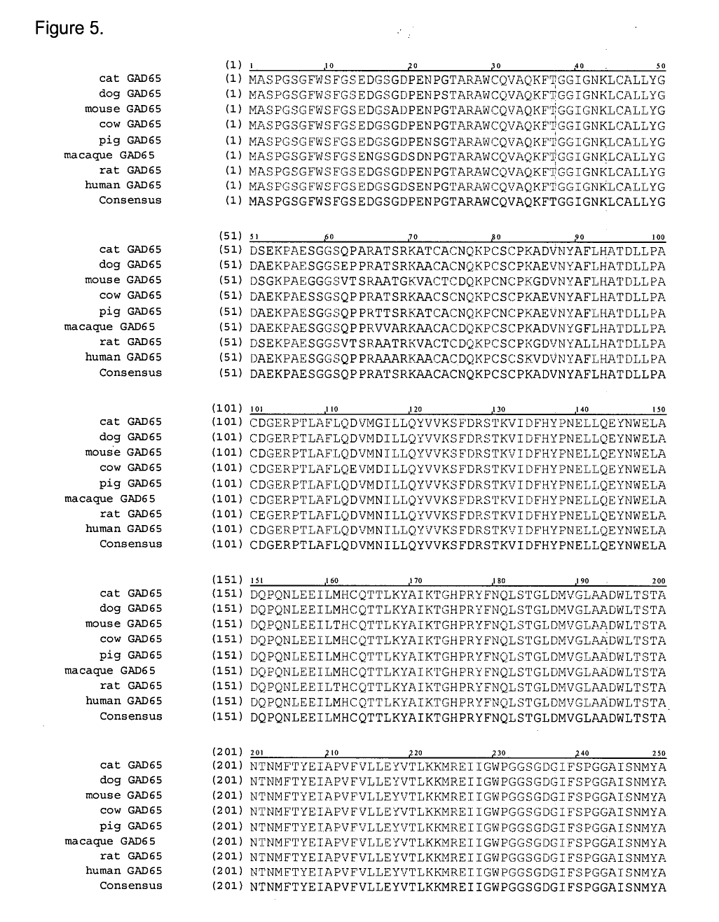

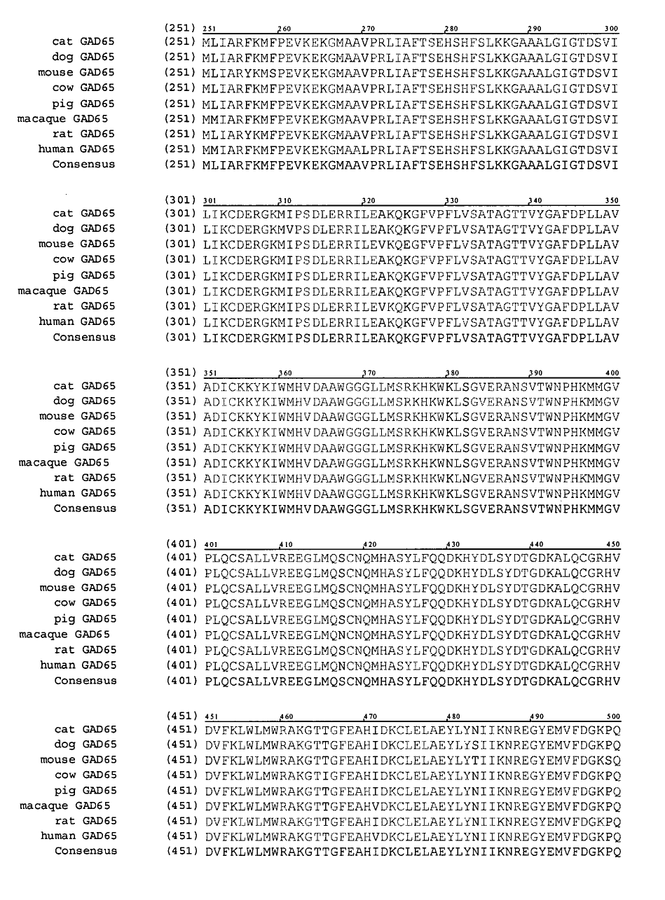

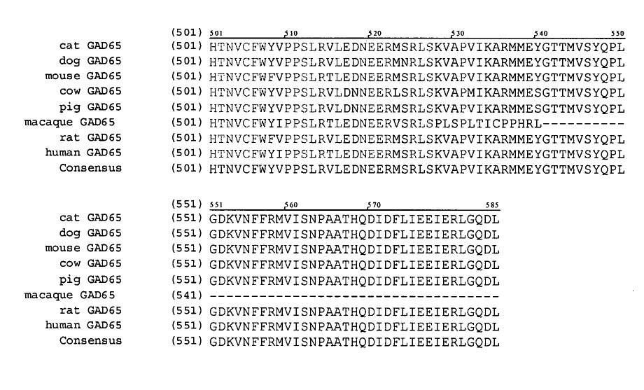

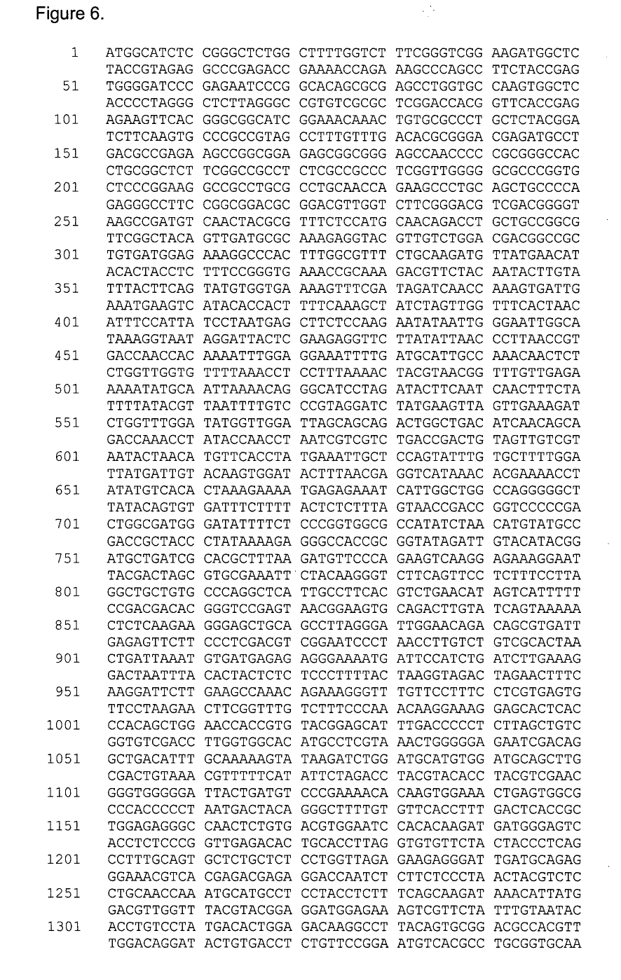

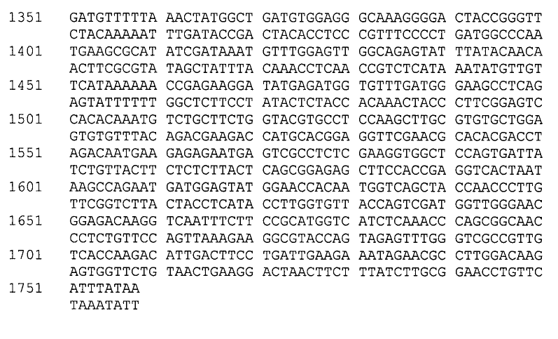

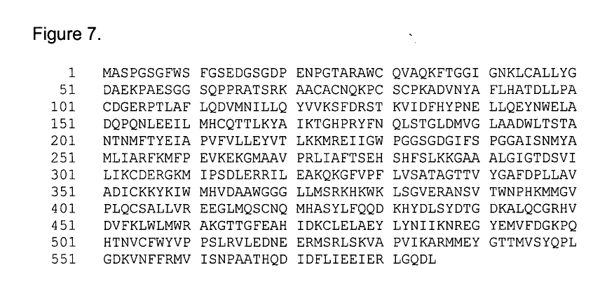

A consensus GAD65 sequence was generated using AlignX software (Vector NTI, Informax) and based on an alignment of the mammalian GAD65 sequences available on GenBank as of April 2006, namely: homo sapiens (human) (SEQ ID NO. 11), mus musculus (mouse) (SEQ ID NO. 6), rattus norvegicus (rat) (SEQ ID NO. 10), canis familiaris (dog) (SEQ ID NO. 5), sus scrofa (pig) (SEQ ID NO. 8), bos taurus (cow) (SEQ ID NO. 7), macaca mulatta (macaque) (SEQ ID NO. 9); as well as felis catus (cat) (SEQ ID NO. 2) GAD65 which was cloned by our laboratory (FIG. 5). The Consensus GAD65 DNA sequence is shown in FIG. 6 (SEQ ID NO. 3). The Consensus GAD65 amino acid sequence is shown in FIG. 7 (SEQ ID NO. 4).

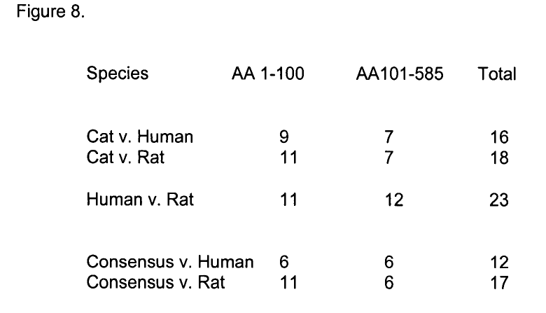

The consensus GAD65 sequence contained number of amino acid changes to human and rat GAD65 (FIG. 8).

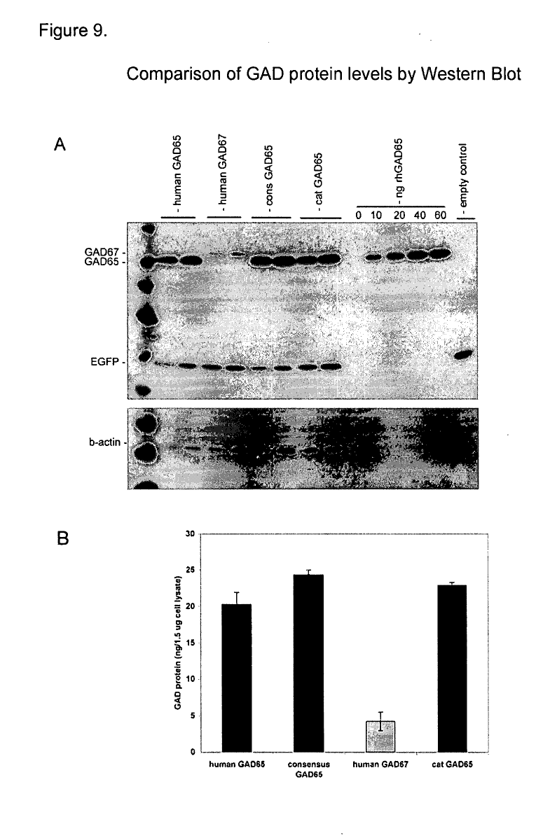

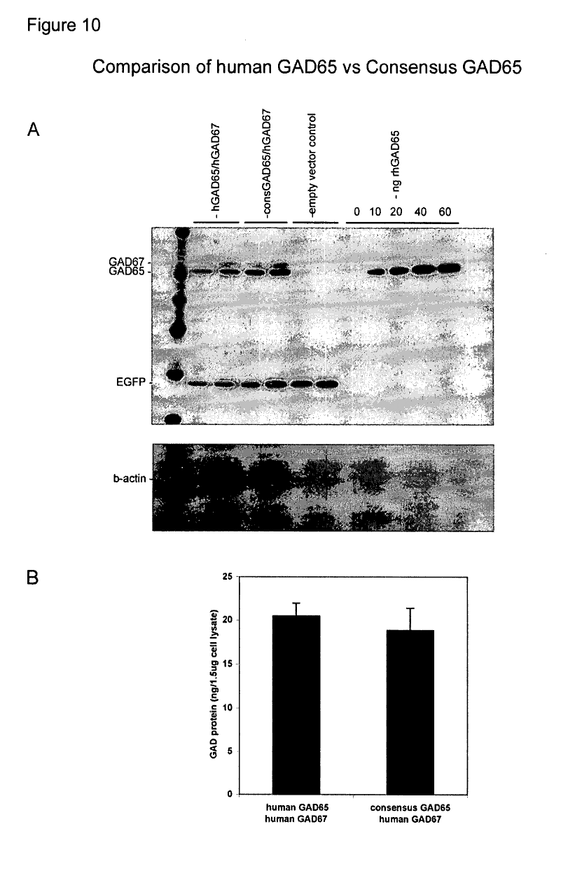

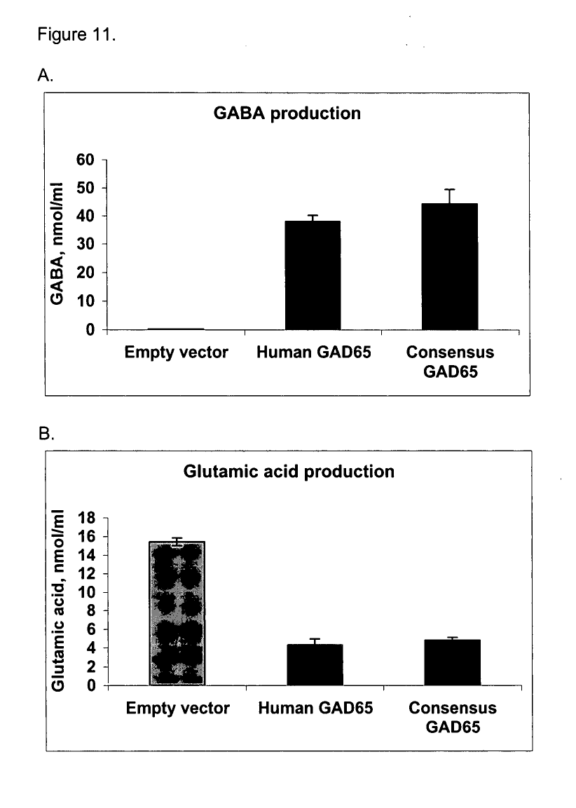

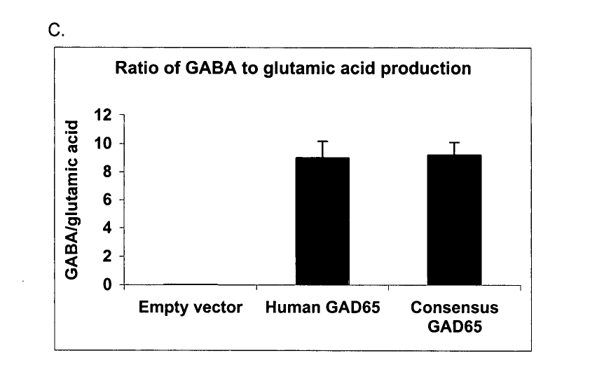

Consensus and Feline GAD65 sequences were cloned into a recombinant adeno-associated virus vector (rAAV) plasmid backbone and transfected into HEK293 cells along with GAD67, in order to evaluate GAD protein expression (FIGS. 9 and 10), and GABA and glutamate release (FIG. 11) compared to human GAD65.

### IV. Binding Molecules

In one aspect, the invention provides molecules that specifically bind to a novel GAD polypeptide or polynucleotide of the invention. The binding molecules include antibodies and antibody fragments.

In one aspect, the invention provides antibodies that specifically bind to the polypeptides of SEQ. I.D. NO:2 or SEQ. I.D. NO:4, or to a molecule that comprises a foregoing component.

In another embodiment, the invention provides antibodies that specifically bind to a polypeptide having substantial homology with SEQ. I.D. NO:2 or SEQ. I.D. NO:4, or to a molecule that comprises a foregoing polypeptide.

In another embodiment, the invention provides antibodies that specifically bind to a component that is a fragment, modification, precursor or successor of Consensus GAD.

In another embodiment, the invention provides antibodies that specifically bind to a Feline or Consensus GAD polypeptide or polynucleotide that is structurally different from a what has been described but has the same (or nearly the same) function or properties, or to a molecule that comprises a foregoing component.

Certain antibodies that specifically bind polypeptide markers, metabolite markers or polynucleotide markers of the invention already may be known and/or available for purchase from commercial sources. In any event, the antibodies of the invention may be prepared by any suitable means known in the art. For example, antibodies may be prepared by immunizing an animal host with a marker or an immunogenic fragment thereof (conjugated to a carrier, if necessary). Adjuvants (e.g., Freund's adjuvant) optionally may be used to increase the immunological response. Sera containing polyclonal antibodies with high affinity for the antigenic determinant can then be isolated from the immunized animal and purified.

Alternatively, antibody-producing tissue from the immunized host can be harvested and a cellular homogenate prepared from the organ can be fused to cultured cancer cells. Hybrid cells which produce monoclonal antibodies specific for a marker can be selected. Alternatively, the antibodies of the invention can be produced by chemical synthesis or by recombinant expression. For example, a polynucleotide that encodes the antibody can be used to construct an expression vector for the production of the antibody. The antibodies of the present invention can also be generated using various phage display methods known in the art.

Antibodies that specifically bind markers of the invention can be used, for example, in methods for detecting Consensus GAD polypeptides or polynucleotides using methods and techniques well-known in the art. In some embodiments, for example, the antibodies are conjugated to a detection molecule or moiety (e.g., a dye, and enzyme) and can be used in ELISA or sandwich assays to detect markers of the invention.

In another embodiment, antibodies against a polypeptide or polynucleotide of the invention can be used to assay a tissue sample (e.g., a thin cortical slice) for the marker. The antibodies can specifically bind to the marker, if any, present in the tissue sections and allow the localization of the marker in the tissue. Similarly, antibodies labeled with a radioisotope may be used for in vivo imaging or treatment applications.

### V. Methods for Detecting

The markers of the invention may be detected by any method known to those of skill in the art, including without limitation LC-MS, GC-MS, immunoassays, hybridization and enzyme assays. The detection may be quantitative or qualitative. A wide variety of conventional techniques are available, including mass spectrometry, chromatographic separations, 2-D gel separations, binding assays (e.g., immunoassays), competitive inhibition assays, and so on. Any effective method in the art for measuring the present/absence, level or activity of a metabolite, polypeptide or polynucleotide is included in the invention. It is within the ability of one of ordinary skill in the art to determine which method would be most appropriate for measuring a specific marker. Thus, for example, a ELISA assay may be best suited for use in a physician's office while a measurement requiring more sophisticated instrumentation may be best suited for use in a clinical laboratory. Regardless of the method selected, it is important that the measurements be reproducible.

The markers of the invention can be measured by mass spectrometry, which allows direct measurements of analytes with high sensitivity and reproducibility. A number of mass spectrometric methods are available. Electrospray ionization (ESI), for example, allows quantification of differences in relative concentration of various species in one sample against another; absolute quantification is possible by normalization techniques (e.g., using an internal standard). Matrix-assisted laser desorption ionization (MALDI) or the related SELDI® technology (Ciphergen, Inc.) also could be used to make a determination of whether a marker was present, and the relative or absolute level of the marker. Mass spectrometers that allow time-of-flight (TOF) measurements have high accuracy and resolution and are able to measure low abundant species, even in complex matrices like serum or CSF.

For protein markers, quantification can be based on derivatization in combination with isotopic labeling, referred to as isotope coded affinity tags (“ICAT”). In this and other related methods, a specific amino acid in two samples is differentially and isotopically labeled and subsequently separated from peptide background by solid phase capture, wash and release. The intensities of the molecules from the two sources with different isotopic labels can then be accurately quantified with respect to one another.

In addition, one- and two-dimensional gels have been used to separate proteins and quantify gels spots by silver staining, fluorescence or radioactive labeling. These differently stained spots have been detected using mass spectrometry, and identified by tandem mass spectrometry techniques.

In a preferred embodiment, the markers are measured using mass spectrometry in connection with a separation technology, such as liquid chromatography-mass spectrometry or gas chromatography-mass spectrometry. In particular, coupling reverse-phase liquid chromatography to high resolution, high mass accuracy ESI time-of-flight (TOF) mass spectroscopy allows spectral intensity measurement of a large number of biomolecules from a relatively small amount of any complex biological material. Analyzing a sample in this manner allows the marker (characterized by a specific RT and m/z) to be determined and quantified.

As will be appreciated by one of skill in the art, many other separation technologies may be used in connection with mass spectrometry. For example, a wide selection of separation columns is commercially available. In addition, separations may be performed using custom chromatographic surfaces (e.g., a bead on which a marker specific reagent has been immobilized). Molecules retained on the media subsequently may be eluted for analysis by mass spectrometry.

Analysis by liquid chromatography-mass spectrometry produces a mass intensity spectrum, the peaks of which represent various components of the sample, each component having a characteristic mass-to-charge ratio (m/z) and retention time (RT). The presence of a peak with the m/z and RT of a marker indicates that the marker is present. The peak representing a marker may be compared to a corresponding peak from another spectrum (e.g., from a control sample) to obtain a relative measurement. Any normalization technique in the art (e.g., an internal standard) may be used when a quantitative measurement is desired. “Deconvoluting” software is available to separate overlapping peaks. The retention time depends to some degree on the conditions employed in performing the liquid chromatography separation. The preferred conditions, those used to obtain the retention times that appear in the Tables, are set forth in the Example. The mass spectrometer preferably provides high mass accuracy and high mass resolution. The mass accuracy of a well-calibrated Micromass TOF instrument, for example, is reported to be approximately 2 mDa, with resolution m/Δm exceeding 5000.

In other preferred embodiments, the level of the markers may be determined using a standard immunoassay, such as sandwiched ELISA using matched antibody pairs and chemiluminescent detection. Commercially available or custom monoclonal or polyclonal antibodies are typically used. However, the assay can be adapted for use with other reagents that specifically bind to the marker. Standard protocols and data analysis are used to determine the marker concentrations from the assay data.

A number of the assays discussed above employ a reagent that specifically binds to the marker. Any molecule that is capable of specifically binding to a marker is included within the invention. In some embodiments, the binding molecules are antibodies or antibody fragments. In other embodiments, the binding molecules are non-antibody species. Thus, for example, the binding molecule may be an enzyme for which the marker is a substrate. The binding molecules may recognize any epitope of the targeted markers.

As described above, the binding molecules may be identified and produced by any method accepted in the art. Methods for identifying and producing antibodies and antibody fragments specific for an analyte are well known. Examples of other methods used to identify the binding molecules include binding assays with random peptide libraries (e.g., phage display) and design methods based on an analysis of the structure of the marker.

The markers of the invention, especially the metabolite markers, also may be detected or measured using a number of chemical derivatization or reaction techniques known in the art. Reagents for use in such techniques are known in the art, and are commercially available for certain classes of target molecules.

Finally, the chromatographic separation techniques described above also may be coupled to an analytical technique other than mass spectrometry such as fluorescence detection of tagged molecules, NMR, capillary UV, evaporative light scattering or electrochemical detection.

Measurement of the relative amount of an RNA or protein marker of the invention may be by any method known in the art (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989; and Current Protocols in Molecular Biology, eds. Ausubel et al. John Wiley & Sons: 1992). Typical methodologies for RNA detection include RNA extraction from a cell or tissue sample, followed by hybridization of a labeled probe (e.g., a complementary polynucleotide) specific for the target RNA to the extracted RNA, and detection of the probe (e.g., Northern blotting). Typical methodologies for protein detection include protein extraction from a cell or tissue sample, followed by hybridization of a labeled probe (e.g., an antibody) specific for the target protein to the protein sample, and detection of the probe. The label group can be a radioisotope, a fluorescent compound, an enzyme, or an enzyme co-factor. Detection of specific protein and polynucleotides may also be assessed by gel electrophoresis, column chromatography, direct sequencing, or quantitative PCR (in the case of polynucleotides) among many other techniques well known to those skilled in the art.

Detection of the presence or number of copies of all or a part of a marker gene of the invention may be performed using any method known in the art. Typically, it is convenient to assess the presence and/or quantity of a DNA or cDNA by Southern analysis, in which total DNA from a cell or tissue sample is extracted, is hybridized with a labeled probe (e.g., a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

Polynucleotide similarity can be evaluated by hybridization between single stranded nucleic acids with complementary or partially complementary sequences. Such experiments are well known in the art.

### VI. Neurodegenerative Diseases

Generally, the methods and compositions of the invention can be used to change cells from a first phenotypic state to second phenotypic state. The invention also pertains to delivering Feline or Consensus GAD to regions of the brain associated with a particular disease or disorder. These regions vary according to the neurodegenerative disease, but are well know to the skilled artisan. For example, the region of the brain associated with Parkinson's Disease (PD) can be the Subthalamic Nucleus (STN), while the region of the brain associated with epilepsy can be the hippocampus. It is also to be understood that the various regions of the brain treated with DBS can also be treated with the methods and compositions of the invention.

In particular, the invention pertains to GAD gene transfer into glutamatergic excitatory neurons which leads to an inhibitory bias with altered network activity. This phenotypic shift provides strong neuroprotection and demonstrates there is plasticity between excitatory and inhibitory neurotransmission in the mammalian brain that results in a therapeutic effect. This alteration form a first state to a second state can be used in a umber of neurodegenerative disorders such as those described below.

### (a) Parkinson's Disease

Parkinson's disease is associated with a disturbances of posture, locomotion, facial expression or speech. The manifestations may be asymmetric, e.g., a slight tremor of the fingers on one hand at rest, and then become bilateral. Symptoms of Parkinson's disease are caused by loss of nerve cells in the pigmented substantia nigra pars compacta (SNPC) and the locus ceruleus in the midbrain. The stratium or corpus stratium is a structure in the cerebral hemispheres consisting of two basal ganglia (the caudate nucleus and the putamen) and the fiber of the internal capsule that separate them. Parkinson's disease in humans primarily effects the subcortical structures, especially the substantia nigra and the locus ceruleus. It is characterized by the loss of dopamine neurons in the substantia nigra, which have the basal ganglia as their major target organ. Cell loss also occurs in the globus pallidus and putamen.

Parkinson's disease is also associated with eosinophilic intraneural inclusion granules (Lewy bodies) which are present in the basal ganglia, brainstem, spinal cord, and sympathetic ganglia. The pars compacta neurons of the substantia nigra (SN) provide dopaminergic input into the stratium, which is part of the basal ganglia. These dopaminergic neurons modulate a monosynaptic gamma-aminobutyric acid (GABA) inhibitory output in the globus pallidus interna and pars reticulata of the substantia nigra. In Parkinson's disease, loss of dopaminergic cells in the substantia nigra leads to stratial dopamine depletion. This loss of dopamine alters the activity of neurons within the basal ganglia circuitry, including excessive firing and activity of these cells.

The motor abnormalities of Parkinson's disease (PD) are caused by alterations in basal ganglia network activity, including disinhibition of the subthalamic nucleus (STN), and excessive activity of the major output nuclei. Using adeno-associated viral vector-mediated somatic cell gene transfer, glutamic acid decarboxylase (GAD) was expressed, the enzyme that catalyzes synthesis of the neurotransmitter GABA, in excitatory glutamatergic neurons of the STN in an in vivo animal model. The transduced neurons, when driven by electrical stimulation, produced mixed inhibitory responses associated with GABA release. This phenotypic shift resulted in strong neuroprotection of nigral dopamine neurons and rescue of the parkinsonian behavioral phenotype. This strategy suggests that there is plasticity between excitatory and inhibitory neurotransmission in the mammalian brain that could be exploited for therapeutic benefit (See Luo et al., (2002) Science 298: 425-429).

Accordingly, a region of the brain associated with Parkinson's disease can be inhibited, reduced, treated, or altered from a first phenotypic state to a second phenotypic state using the methods and compositions of the invention. In particular, a vector comprising a therapeutic agent, e.g., a nucleotide sequence encoding the novel GAD of the invention, can be delivered to the site of domaminergic cell loss or other regions of the basal ganglia and output nuclei. In one embodiment, the vector comprising a therapeutic agent can be delivered to the subthalamic nucleus (SN). In another embodiment, the vector comprising a therapeutic agent can be delivered to the substantia nigra pars reticulata (SNPR).

### (b) Alzheimer's Disease

Alzheimer's disease is characterized by the gradual loss of intellectual capabilities. Post-mortem examination of the brain shows a generalized atrophy. There are extensive histological changes in Alzheimer's disease dominated by the presence of intracellular amyloid plaques and neurofibrillary tangles. Plaques and tangles are rare, however, in the basal ganglia and substantia nigra. Many specimens from Alzheimer's disease patients demonstrate a loss of pigmentation in the area of the locus ceruleus, which is a major source of noradrenergic synthesis in the brain. Accordingly, a region of the brain associated with Alzheimer's disease can be inhibited, reduced, treated, or altered from a first phenotypic state to a second phenotypic state using the methods and compositions of the invention.

### (c) Epilepsy

Epileptic seizures are the outward manifestation of excessive and/or hypersynchronous abnormal activity of neurons in the cerebral cortex. Seizures are usually self limiting. Many types of seizures occur. The behavioral features of a seizure reflect function of the portion of the cortex where the hyper activity is occurring. Seizures can be generalized, appearing to involve the entire brain simultaneously. Generalized seizures can result in the loss of conscious awareness only and are then called absence seizures (previously referred to as “petit mal”). Alternatively, the generalized seizure may result in a convulsion with tonic-clonic contractions of the muscles (“grand mall” seizure). Some types of seizures, partial seizures, begin in one part of the brain and remain local. The person may remain conscious throughout the seizure. If the person loses consciousness the seizure is referred to as a complex partial seizure.

Simple partial seizures include autonomic and mental symptoms and sensory symptoms such as olfaction, audition, or vision, sometimes concomitant with symptoms of experiences such as deja-vu and jamais-vu. Complex partial seizures often exhibit motion stopping followed by eating-function automatism, and are divided into amygdala-hippocampus seizures and lateral temporal lobe seizures according to localization. In the case of temporal lobe epilepsy, 70-80% of the seizures are hippocampus seizures, in which aura, motion stopping, lip automatism, and clouding of consciousness are successively developed to result in amnesia. When the focus is in the amygdala, autonomic symptoms such as dysphoria in the epigastrium are manifested; phobia; and olfactory hallucination. Lateral temporal lobe seizures include auditory illusion, hallucination, and a dreamy state, and disturbance of speech when the focus is in the dominant hemisphere. Temporal lobe epilepsy exhibits a long-term psychosis-like state in addition to other symptoms and recognition-and-memory disorder more frequently than do other epilepsies. Treatment of temporal lobe epilepsy is carried out through pharmacotherapy employing a maximum dose of a combination of drugs, or through surgical treatment. A complex partial seizure is a partial seizure with impairment of consciousness, and is similar to a seizure that has conventionally been called a psycho-motor seizure or a seizure associated with temporal lobe epilepsy.

The neuromechanism responsible for seizures includes the amygdala, the hippocampus, the hypothalamus, the parolfactory cortex, etc., in addition to the frontal and temporal lobes. The seizures typically last 1-2 minutes or slightly longer, and the onset and cessation of the seizures are not abrupt but gradual.

The existence of a system which can control the propagation and/or the generation of different kinds of seizures is known. The involvement of the substantia nigra, a particular portion of the brain considered to be part of neural circuitry referred to as the basal ganglia (See e.g., Depaulis, et al. (1994) Prog. Neurobiology, 42: 33-52). The inhibition of the substantia nigra will increase the threshold for seizure.

The neural connections that make up the basal ganglia are also important in epilepsy. These connections are reviewed by Alexander et. al. (Alexander, et al. Prog. Brain Res. 85: 119-146). The substantia nigra receives input from the subthalamic nucleus (STN) which is excitatory and involves glutamate as the neurotransmitter conveying information at the synapse. Bergman et al. have shown that a lesion of the subthalamic nucleus will reduce the inhibitory output of the internal segment of the globus pallidus and substantia nigra reticulata (SN) (Bergman, et al (1990), Science, 249: 1436-1438). The subthalamic nucleus receives input from the external segment of the globus pallidus (GPe). This input is inhibitory using GABA as a transmitter substance. Hence, increased activity of the neurons in GPe will increase inhibition of neurons in the subthalamic nucleus which will reduce the excitation of neurons in the substantia nigra.

Accordingly, a region of the brain associated with epilepsy can be inhibited, reduced, treated, or altered from a first phenotypic state to a second phenotypic state using the methods and compositions of the invention. The invention is intended to include all regions of the brain associated with epilepsy. Such regions of the brain include, but are not limited to, the hippocampus, amygdala, and hypothalamus. In a preferred embodiment, the vector carrying the GAD gene is delivered to the hippocampus. Also within the scope of the invention are regions for treatment of epilepsy via DBS, such as cerebellum, caudate, thalamus, mamillary nuclei, anterior nucleus of the thalamus, centromedian nucleus of the thalamus, and subthalamic nucleus.

The kainate model is an epileptic model in which kainic acid, which is one of the excitatory amino acids found in the brain, is injected to nuclei (amygdala, hippocampus, etc.) in the limbic system in an microamount to induce focal epilepsy. The kainate model serves as a model for an epileptic seizure; more particularly, as a model for status epilepticus induced from the limbic system in an acute phase, and as a model for evolution of a spontaneous limbic seizure to a secondary generalized seizure in a chronic phase. The kainate model may also be used as a cortex epilepsy model through injection of kainic acid to the cortex (sensory motor field).

The methods and compositions of the invention can be used to be used to inhibit, reduce, or treat seizures that include, but are not limited to, tonic seizures, tonic-clonic seizures, atypical absence seizures, atonic seizures, myoclonic seizures, clonic seizures, simple partial seizures, complex partial seizures, and secondary generalized seizures.

### (d) Metabolic Disorders

### (i) Obesity

The methods and composition of the invention can also be used to treat or modify obesity in a subject. Mouse models for obesity are known in that art, for example, obese-diabetic mice (ob/ob), and obese-diabetic (db/db) mice from the Jackson Laboratories (Bar Harbor, Me.). (See e.g. Collins et al. (1996) J Biol Chem 271:9437-9440; Darling (1996) Curr Opin Genet Dev 6:289-294; Andersson (1996) Ann. Med. 28:5-7). These animal models can be used to assess the effect of GAD on weight gain, particularly by delivering GAD to the hypothalamus region of the brain. The hypothalamus plays a significant role in obesity. Augmentation of GABA function from neurons within the hypothalamus can result in alteration of metabolic behavior (Boulis et al. (2002) AANS meeting, Chicago (abstract)). Accordingly, a region of the brain associated with obesity can be inhibited, reduced, treated, or altered from a first phenotypic state to a second phenotypic state using the methods and compositions of the invention.

### (ii) Diabetes

A summary of insulin-dependent diabetes mellitus and its animal models is described by Wong et al. (1999) Curr Opin Immunol 11:643-647. Glutamic acid decarboxylase (GAD) has been associated with diabetes (Baekkeskov et al. (1990) Nature 347:151-156). These models can be used to investigate the effect of GAD on diabetes in a animal. A region of the brain associated with diabetes can be inhibited, reduced, treated, or altered from a first phenotypic state using the methods and compositions of the invention.

### (e) Pain

The methods and compositions of the invention can also be used to reduce pain by delivering GAD to a region of the brain associated with pain. For example, in Jasmin et al., GABA neurotransmission in the rostral agranular insular cortex (RAIC) of freely moving rats, was altered by locally increasing GABA using two methods: (a) an enzyme inhibitor; and (b) a double-cassette-defective herpes simplex virus vector. Use of gene transfer mediated by a viral vector produced lasting analgesia in the rats by enhancing the descending inhibition of spinal nociceptive neurons (Jasmin et al. (2003) Nature, 424:316-320). A region of the brain associated with pain can be inhibited, reduced, treated, or altered from a first phenotypic state using the methods and compositions of the invention.

### (f) Visual Cortical Function

The methods and compositions of the invention can also be used to improve visual cortical function be delivering GAD, and to subsequently alter GABA levels in a region of the brain associated with vision. For example, alteration of GABA levels, in a region of the visual cortex (VI) of aged primates can result in improved acuity, improved orientation and direction selectivity, decreased spontaneous activity and an increased ability to signal visual stimuli (Levanthal et al. (2003) Science 300:812-815). Accordingly, a region of the brain associated with vision can be inhibited, reduced, treated, or altered from a first phenotypic state using the methods and compositions of the invention.

### (g) Other Degenerative Diseases

This invention also relates to compositions and methods of treatment of other degenerative disorders. These include, but are not limited to the following: head and spinal cord trauma; cardiac cell death due to ischemia; tissue and organ death due to transplant rejection; and hearing loss due to autotoxicity.

### VII. Vectors

The vectors of the invention can be delivered to the cells of the central nervous system by using viral vectors or by using non-viral vectors. In a preferred embodiment, the invention uses adeno-associated viral vectors comprising the a nucleotide sequence encoding the novel GAD of the invention (Feline and/or Consensus GAD) for gene delivery. AAV vectors can be constructed using known techniques to provide at least the operatively linked components of control elements including a transcriptional initiation region, a exogenous nucleic acid molecule, a transcriptional termination region and at least one post-transcriptional regulatory sequence. The control elements are selected to be functional in the targeted cell. The resulting construct which contains the operatively linked components is flanked at the 5′ and 3′ region with functional AAV ITR sequences.

The nucleotide sequences of AAV ITR regions are known. The ITR sequences for AAV-2 are described, for example by Kotin et al. (1994) Human Gene Therapy 5:793-801; Berns “Parvoviridae and their Replication” in Fundamental Virology, 2nd Edition, (B. N. Fields and D. M. Knipe, eds.) The skilled artisan will appreciate that AAV ITR's can be modified using standard molecular biology techniques. Accordingly, AAV ITRs used in the vectors of the invention need not have a wild-type nucleotide sequence, and may be altered, e.g. by the insertion, deletion or substitution of nucleotides. Additionally, AAV ITRs may be derived from any of several AAV serotypes, including but not limited to, AAV-1, AAV-2, AAV-3, AAV-4, AAV-5, AAVX7, AAV-8 and the like. Furthermore, 5′ and 3′ ITRs which flank a selected nucleotide sequence in an AAV expression vector need not necessarily be identical or derived from the same AAV serotype or isolate, so long as the ITR's function as intended, i.e., to allow for excision and replication of the bounded nucleotide sequence of interest when AAV rep gene products are present in the cell.

The skilled artisan can appreciate that regulatory sequences can often be provided from commonly used promoters derived from viruses such as, polyoma, Adenovirus 2, cytomegalovirus and Simian Virus 40. Use of viral regulatory elements to direct expression of the protein can allow for high level constitutive expression of the protein in a variety of host cells. Ubiquitously expressing promoters can also be used include, for example, the early cytomegalovirus promoter Boshart et al. (1985) Cell 41:521-530, herpesvirus thymidine kinase (HSV-TK) promoter (McKnight et al. (1984) Cell 37: 253-262), Z-actin promoters (e.g., the human Z-actin promoter as described by Ng et al. (1985) Mol. Cell. Biol. 5: 2720-2732) and colony stimulating factor-1 (CSF-1) promoter (Ladner et al. (1987) EMBO J. 6: 2693-2698).

Alternatively, the regulatory sequences of the AAV vector can direct expression of the gene preferentially in a particular cell type, i.e., tissue-specific regulatory elements can be used. Non-limiting examples of tissue-specific promoters which can be used include, central nervous system (CNS) specific promoters such as, neuron-specific promoters (e.g., the neurofilament promoter; Byrne and Ruddle (1989) Proc. Natl. Acad. Sci. USA 86:5473-5477) and glial specific promoters (Morii et al. (1991) Biochem. Biophys Res. Commun. 175: 185-191). Preferably, the promoter is tissue specific and is essentially not active outside the central nervous system, or the activity of the promoter is higher in the central nervous system that in other systems. For example, a promoter specific for the spinal cord, brainstem, (medulla, pons, and midbrain), cerebellum, diencephalon (thalamus, hypothalamus), telencephalon (corpus stratium, cerebral cortex, or within the cortex, the occipital, temporal, parietal or frontal lobes), or combinations, thereof. The promoter may be specific for particular cell types, such as neurons or glial cells in the CNS. If it is active in glial cells, it may be specific for astrocytes, oligodentrocytes, ependymal cells, Schwann cells, or microglia. If it is active in neurons, it may be specific for particular types of neurons, e.g. motor neurons, sensory neurons, or interneurons. Preferably, the promoter is specific for cells in particular regions of the brain, for example, the cortex, stratium, nigra and hippocampus.

Suitable neuronal specific promoters include, but are not limited to, neuron specific enolase (NSE) (Olivia et al. (1991) Genomics 10: 157-165, GenBank Accession No: X51956), and human neurofilament light chain promoter (NEFL) (Rogaev et al. (1992) Hum. Mol. Genet. 1: 781, GenBank Accession No: L04147). Glial specific promoters include, but are not limited to, glial fibrillary acidic protein (GFAP) promoter (Morii et al. (1991) Biochem. Biophys Res. Commun. 175: 185-191, GenBank Accession No: M65210), S100 promoter (Morii et al. (1991) Biochem. Biophys Res. Commun. 175: 185-191, GenBank Accession No: M65210) and glutamine synthase promoter (Van den et al. (1991) Biochem. Biophys. Acta. 2:249-251, GenBank Accession No: X59834). In a preferred embodiment, the gene is flanked upstream (i.e., 5′) by the neuron specific enolase (NSE) promoter. In another preferred embodiment, the gene of interest is flanked upstream (i.e., 5′) by the elongation factor 1 alpha (EF) promoter.

The AAV vector harboring the nucleotide sequence encoding a protein of interest, e.g. GAD, and a post-transcriptional regulatory sequence (PRE) flanked by AAV ITRs, can be constructed by directly inserting the nucleotide sequence encoding the protein of interest and the PRE into an AAV genome which has had the major AAV open reading frames (“ORFs”) excised therefrom. Other portions of the AAV genome can also be deleted, as long as a sufficient portion of the ITRs remain to allow for replication and packaging functions. These constructs can be designed using techniques well known in the art. (See, e.g. Lebkowski et al. (1988) Molec. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling et al. (1994) Gene Therapy 1: 165-169; and Zhou et al. (1994) J. Exp. Med. 179:1867-1875).

Alternatively, AAV ITRs can be excised from the viral genome or from an AAV vector containing the same and fused 5′ and 3′ of a selected nucleic acid construct that is present in another vector using standard ligation techniques, such as those described in Sambrook et al, Supra. Several AAV vectors are available from the American Type Culture Collection (“ATCC”) under Accession Numbers 53222, 53223, 53224, 53225 and 53226.

In order to produce recombinant AAV particles, an AAV vector can be introduced into a suitable host cell using known techniques, such as by transfection. A number of transfection techniques are generally known in the art. See, e.g., Graham et al. (1973) Virology, 52:456, Sambrook et al. (1989) Molecular Cloning, a laboratory manual, Cold Spring Harbor Laboratories, N.Y., Davis et al. (1986) Basic Methods in Molecular Biology, Elsevier, and Chu et al. (1981) Gene 13:197. Particularly suitable transfection methods include calcium phosphate co-precipitation (Graham et al. (1973) Virol. 52:456-467), direct micro-injection into cultured cells (Capecchi (1980) Cell 22:479-488), electroporation (Shigekawa et al. (1988) BioTechniques 6:742-751), liposome mediated gene transfer (Mannino et al. (1988) BioTechniques 6:682-690), lipid-mediated transduction (Felgner et al. (1987) Proc. Natl. Acad. Sci. USA 84:7413-7417), and nucleic acid delivery using high-velocity microprojectiles (Klein et al. (1987) Nature 327:70-73).

Suitable host cells for producing recombinant AAV particles include, but are not limited to, microorganisms, yeast cells, insect cells, and mammalian cells, that can be, or have been, used as recipients of a exogenous nucleic acid molecule. Thus, a “host cell” as used herein generally refers to a cell which has been transfected with an exogenous nucleic acid molecule. The host cell includes any eukaryotic cell or cell line so long as the cell or cell line is not incompatible with the protein to be expressed, the selection system chosen or the fermentation system employed. Non-limiting examples include CHO dhfr− cells (Urlaub and Chasin (1980) Proc. Natl. Acad. Sci. USA 77:4216-4220), 293 cells (Graham et al. (1977) J. Gen. Virol. 36: 59) or myeloma cells like SP2 or NS0 (Galfre and Milstein (1981) Meth. Enzymol. 73(B):3-46).

In one embodiment, cells from the stable human cell line, 293 (readily available through, e.g., the ATCC under Accession No. ATCC CRL1573) are preferred in the practice of the present invention. Particularly, the human cell line 293, which is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a particularly convenient platform in which to produce rAAV virions.

Host cells containing the above-described AAV vectors must be rendered capable of providing AAV helper functions in order to replicate and encapsidate the expression cassette flanked by the AAV ITRs to produce recombinant AAV particles. AAV helper functions are generally AAV-derived coding sequences which can be expressed to provide AAV gene products that, in turn, function in trans for productive AAV replication. AAV helper functions are used herein to complement necessary AAV functions that are missing from the AAV vectors. Thus, AAV helper functions include one, or both of the major AAV open reading frames (ORFs), namely the rep and cap coding regions, or functional homologues thereof.

The AAV rep coding region of the AAV genome encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other exogenous) promoters. The Rep expression products are collectively required for replicating the AAV genome. The AAV cap coding region of the AAV genome encodes the capsid proteins VP1, VP2, and VP3, or functional homologues thereof AAV helper functions can be introduced into the host cell by transfecting the host cell with an AAV helper construct either prior to, or concurrently with, the transfection of the AAV vector comprising the expression cassette, AAV helper constructs are thus used to provide at least transient expression of AAV rep and/or cap genes to complement missing AAV functions that are necessary for productive AAV infection. AAV helper constructs lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. (See, e.g. Samulski et al. (1989) J. Virol. 63:3822-3828; and McCarty et al. (1991) J. Virol. 65:2936-2945). A number of other vectors have been described which encode Rep and/or Cap expression products. See, e.g. U.S. Pat. No. 5,139,941.

As a consequence of the infection of the host cell with a helper virus, the AAV Rep and/or Cap proteins are produced. The Rep proteins also serve to duplicate the AAV genome. The expressed Cap proteins assemble into capsids, and the AAV genome is packaged into the capsids. This results the AAV being packaged into recombinant AAV particles comprising the expression cassette. Following recombinant AAV replication, recombinant AAV particles can be purified from the host cell using a variety of conventional purification methods, such as CsCl gradients. The resulting recombinant AAV particles are then ready for use for gene delivery to various cell types.

Alternatively, a vector of the invention is a recombinant AAV pseudotypes wherein the capsids consist of, but are not limited to, combinations of any of the following serotypes: AAV-1 AAV-2, AAV-3, AAV-4, AAV-5, AAV-7, and AAV-8. Such vectors and methods of their production are more completely described in U.S. application Ser. No. 09/804,898, which is hereby incorporated by reference. In a preferred embodiment, the AAV pseudotype is an AAV1/2 pseudotype with a 1:1 ratio of AAV1 and AAV2 VP1,2 and 3 proteins.

Alternatively, a vector of the invention can be a virus other than the adeno-associated virus, or portion thereof, which allows for expression of a nucleic acid molecule introduced into the viral nucleic acid. For example, replication defective retroviruses, adenoviruses, herpes simplex virus, and lentivirus can be used. Protocols for producing recombinant retroviruses and for infecting cells in vitro or in vivo with such viruses can be found in Current Protocols in Molecular Biology, Ausubel et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include Crip, Cre, 2 and Am. The genome of adenovirus can be manipulated such that it encodes and expresses the protein of interest but is inactivated in terms of its ability to replicate in a normal lytic viral life cycle. See e.g. Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art.

Alternatively, the vector can be delivered using a non-viral delivery system. This includes delivery of the vector to the desired tissues in colloidal dispersion systems that include, for example, macromolecule complexes, nanocapsules, microspheres, beads, and lipid-based systems including oil-in-water emulsions, micelles, mixed micelles, and liposomes.

Liposomes are artificial membrane vesicles which are useful as delivery vehicles in vitro and in vivo. In order for a liposome to be an efficient gene transfer vehicle, the following characteristics should be present: (1) encapsulation of the genetic material at high efficiency while not compromising the biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al. (1988) Biotechniques, 6:682). Examples of suitable lipids liposomes production include phosphatidyl compounds, such as phosphatidylglycerol, phosphatidylcholine, phosphatidylserine, phosphatidylethanolamine, sphingolipids, cerebrosides, and gangliosides. Additional examples of lipids include, but are not limited to, polylysine, protamine, sulfate and 3b-[N-(N′,N′ dimethylaminoethane) carbamoyl] cholesterol.

Alternatively, the vector can be coupled with a carrier for delivery Exemplary and preferred carriers are keyhole limpet hemocyanin (KLH) and human serum albumin. Other carriers may include a variety of lymphokines and adjuvants such as INF, IL-2, IL-4, IL-8 and others. Means for conjugating a peptide to a carrier protein are well known in the art and include glutaraldehyde, m-maleimidobenzoyl-N-hydroxysuccinimide ester, carbodiimyde and bis-biazotized benzidine. The vector can be conjugated to a carrier by genetic engineering techniques that are well known in the art. (See e.g. U.S. Pat. Nos. 4,608,251; 4,601,903; 4,599,231; 4,599,230; 4,596,792; and 4,578,770).

In one embodiment, particle-mediated delivery using a gene-gun can be used as a method to deliver the vector. Suitable particles for gene gun-based delivery of include gold particles. In one embodiment, the vector can be delivered as naked DNA. Gene gun based delivery is described, for example by, Braun et al. (1999) Virology 265:46-56; Drew et al. (1999) Vaccine 18:692-702; Degano et al. (1999) Vaccine 18:623-632; and Robinson (1999) Int J Mol Med 4:549-555; Lai et al. (1998) Crit Rev Immunol 18:449-84; See e.g. Accede et al. (1991) Nature 332: 815-818; and Wolff et al. (1990) Science 247:1465-1468 Murashatsu et al., (1998) Int. J. Mol. Med. 1: 55-62; Agracetus et al. (1996) J. Biotechnol. 26: 37-42; Johnson et al. (1993) Genet. Eng 15: 225-236). Also within the scope of the invention is the delivery of the vector in one or more combinations of the above delivery methods.

### VIII. Vector Delivery Systems

Delivery systems include methods of in vitro, in vivo and ex vivo delivery of the vector. For in vivo delivery, the vector can be administered to a subject in a pharmaceutically acceptable carrier. The term “pharmaceutically acceptable carrier”, as used herein, refers to any physiologically acceptable carrier for in vivo administration of the vectors of the present invention. Such carriers do not induce an immune response harmful to the individual receiving the composition, and are discussed in section _.

In one embodiment, vector can be distributed throughout a wide region of the CNS, by injecting the vector into the cerebrospinal fluid, e.g. by lumbar puncture (See e.g., Kapadia et al. (1996) Neurosurg 10: 585-587).

Alternatively, precise delivery of the vector into specific sites of the brain, can be conducted using stereotactic microinjection techniques. For example, the subject being treated can be placed within a stereotactic frame base (MRI-compatible) and then imaged using high resolution MRI to determine the three-dimensional positioning of the particular region to be treated. The MRI images can then be transferred to a computer having the appropriate stereotactic software, and a number of images are used to determine a target site and trajectory for antibody microinjection. The software translates the trajectory into three-dimensional coordinates that are precisely registered for the stereotactic frame. In the case of intracranial delivery, the skull will be exposed, burr holes will be drilled above the entry site, and the stereotactic apparatus used to position the needle and ensure implantation at a predetermined depth. The vector can be delivered to regions, such as the cells of the spinal cord, brainstem, (medulla, pons, and midbrain), cerebellum, diencephalon (thalamus, hypothalamus), telencephalon (corpus stratium, cerebral cortex, or within the cortex, the occipital, temporal, parietal or frontal lobes), or combinations, thereof. In another preferred embodiment, the vector is delivered using other delivery methods suitable for localized delivery, such as localized permeation of the blood-brain barrier. Particularly preferred delivery methods are those that deliver the vector to regions of the brain that require modification.

Modification as used herein refers to a change in the cellular activity in the region of the brain injected with the vector. The change in cellular activity can result from changing the expression, or production of genes responsible for stimulating a cell. For example, delivery of a vector comprising a nucleotide sequence encoding GAD, to a region of the brain that is overstimulated, such as the basal ganglia. In particular, delivery of the vector to the STN which are overactive in diseases such as Parkinson's, will result in expression of GAD in this region. While not being required to provide a mechanism of action, the expression of GAD in the STN results in production of GABA within the STN cells, the STN cells release GABA locally such that the released GABA binds to GABA-A and GABA-B receptors on the STN cell surface. GABA binding to the GABA receptors results in a reduction in cell stimulation, thereby reducing overactivity in the STN cells and prevent neuronal destruction.

Other examples of Vectors, Compositions and Methods of gene delivery can be found in U.S. Pat. Nos. 7,250,299, 7,244,423, 7,238,674, 6,503,888, 6,162,796, 6,040,172, all of which are hereby incorporated by reference.

### IX. Pharmaceutical Compositions and Pharmaceutical Administration

Another aspect of the invention provides compositions comprising a polypeptide, or polynucleotide of the invention, a binding molecule that is specific for a Consensus GAD polypeptide or polynucleotide (e.g., an antibody), an inhibitor of a Consensus GAD polypeptide or polynucleotide, or other molecule that can increase or decrease the level or activity of a Consensus GAD polypeptide or polynucleotide marker. Such compositions may be pharmaceutical compositions formulated for use as a therapeutic.

In one embodiment, the invention provides a composition that comprises a novel GAD polypeptide or polynucleotide of the invention, such as those described in SEQ. I.D. NO:1, SEQ. I.D. NO:2, SEQ. I.D. NO:3, or SEQ. I.D. NO:4, or polypeptides having substantial homology with one of the aforementioned Consensus GAD polypeptides.

Alternatively, the invention provides a composition that comprises a component that is a fragment, modification, precursor or successor of a novel GAD polynucleotide or polypeptide of SEQ. I.D. NO:1, SEQ. I.D. NO:2, SEQ. I.D. NO:3, or SEQ. I.D. NO:4 or to a molecule that comprises a foregoing component.

In another embodiment, the invention provides a composition that comprises a polypeptide or metabolite that is structurally different from a component specifically identified in SEQ. I.D. NO:1, SEQ. I.D. NO:2, SEQ. I.D. NO:3, or SEQ. I.D. NO:4, but has the same function or properties, or a molecule that comprises a foregoing component.

In another embodiment, the invention provides a composition that comprises a polynucleotide that binds to a GAD polypeptides of the invention or a molecule that comprises a foregoing polynucleotide.

In another embodiment, the invention provides a composition that comprises an antibody that specifically binds to a novel GAD polypeptide, or a molecule that comprises a foregoing antibody.

In another embodiment, the invention provides a composition that comprises a modulator of the level or activity of the novel GAD polypeptide of the invention (e.g., an inhibitor of a Consensus GAD or Feline polypeptide, an antisense polynucleotide which is complementary to a polynucleotide that encodes a Consensus or Feline GAD polypeptide), or a molecule that comprises a foregoing modulator.

Such compositions may be pharmaceutical compositions. Typically, a pharmaceutical composition comprises a therapeutically effective amount of an active agent and is formulated with a suitable excipient or carrier. The invention also provides pharmaceutical compositions for the treatment of various disorders including neurological disorders. These compositions may include a protein and/or nucleic acid of the invention, and can be formulated as described herein. Alternately, these compositions may include an antibody which specifically binds to a protein of the invention and/or an antisense polynucleotide which is complementary to a polynucleotide of the invention and can be formulated as described herein.

The pharmaceutical compositions of the invention can be prepared in any suitable manner known in the pharmaceutical art. The carrier or excipient may be a solid, semisolid, or liquid material that can serve as a vehicle or medium for the active ingredient. Suitable carriers or excipients are well known in the art and include, but are not limited to saline, buffered saline, dextrose, water, glycerol, ethanol, and combinations thereof. The pharmaceutical compositions may be adapted for oral, inhalation, parenteral, or topical use and may be administered to the patient in the form of tablets, capsules, aerosols, inhalants, suppositories, solutions, suspensions, powders, syrups, and the like. As used herein, the term “pharmaceutical carrier” may encompass one or more excipients. In preparing formulations of the compounds of the invention, care should be taken to ensure bioavailability of an effective amount of the agent. Suitable pharmaceutical carriers and formulation techniques are found in standard texts, such as Remington's Pharmaceutical Sciences, Mack Publishing Co., Easton, Pa.

The vector of the invention can also be incorporated into pharmaceutical compositions suitable for administration to a subject. A pharmaceutical composition may comprise the vector of the invention and a pharmaceutically acceptable vector carrier. As used herein, “pharmaceutically acceptable vector carrier” includes any and all solvents, dispersion media, coatings, antibacterial and antifungal agents, isotonic and absorption delaying agents, and the like that are physiologically compatible. Examples of pharmaceutically acceptable vector carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, intramuscular). In one embodiment, the vector is administered by intravenous infusion or injection. In another embodiment, the vector is administered by intramuscular or subcutaneous injection. In another embodiment, the vector is administered perorally. In the most preferred embodiment, the vector is delivered to a specific location using stereostatic delivery.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antigen, antibody or antibody portion) in the required amount in an appropriate solvent with one or a combination of ingredients enumerated above, as required, followed by filtered sterilization.

Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The vector of the present invention can be administered by a variety of methods known in the art. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g. Sustained and Controlled Release Drug Delivery Systems, J. R. Robinson, ed., Marcel Dekker, Inc., New York, 1978. The pharmaceutical compositions of the invention may include a “therapeutically effective amount” or a “prophylactically effective amount” of the vectors of the invention. A “therapeutically effective amount” refers to an amount effective, at dosages and for periods of time necessary, to achieve the desired therapeutic result.

A therapeutically effective amount of the vector may vary according to factors such as the disease state, age, sex, and weight of the individual, and the ability of the vector to elicit a desired response in the individual. A therapeutically effective amount is also one in which any toxic or detrimental effects of the vector are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

### EXAMPLES

### Example 1

### Feline GAD65

### Cloning of Feline GAD65

Degenerate primers were designed to highly conserved areas of rat and human GAD65. The primers used were: catGADfor: atggcrtctcckggctcnggctttt and catGADrev: ttayaaatcttgtccnaggcgttckat. PCR was performing using cat brain cDNA as a template, using Accuprime Taq (Invitrogen). Thirty cycles of PCR were performed with a Tₘ of 52° C. on a thermal cycler (MJ research). The resulting 1.76 kb cat cDNA was cloned into pCR2.1-TOPO (Invitrogen) then excised with BamHI and EcoRI and inserted into BamHI and EcoRI of the AAV plasmid pAMWCBA-pl-WPRE-BGH.

### Plasmid Transfection of HEK293 Cells

HEK 293 cells were plated at 5×10⁴ cells/well onto collagen-coated plates, 24 hours prior to transfection. For transfection, 1 μg of the appropriate plasmid DNA (pAM/CBA-hGAD65-WPRE-BGH, pAM/CBA-catGAD65-WPRE-BGH, pAM/CBA-hGAD67-WPRE-BGH) spiked with 0.1 μg of EGFP plasmid was mixed with 50 μl OptiMEM (Invitrogen) in a sterile tube. When GAD65 and GAD67 were co-transfected, 0.5 μg of each plasmid was used. Three microlitres of Optifect (Invitrogen) was mixed with 50 μl OptiMEM in a separate tube, then the contents of the tubes were mixed and incubated at room temperature for 30 min. The plasmid/Optifect solution was then added to a well and mixed by gently pipetting up and down. The transfection mix was incubated with the cells for 48 hours prior to extraction of cytoplasmic proteins.

### Extraction of Cytoplasmic Proteins

200 μl of Mammalian Cell Lysis Reagent (Sigma) was added to each well and incubated at room temperature for 10 mins. The lysates were then transferred to 1.5 ml microfuge tubes and centrifuged at 10,000 g for 10 mins at 4° C. The supernatants were transferred to fresh microfuge tubes and stored at −80° C. for analysis by Western blot.

### Western Blot

The amount of protein in each lysate was quantified using the Total Protein Microassay (Biorad) with BSA from 0 to 25 ng/ml as a standard. Three micrograms of each sample and 5, 10, 20, 40 and 80 ng samples of recombinant human GAD65 (rhGAD65, Diamyd) were processed for SDS-PAGE and Western Blotting analysis according to the Novex NuPage western blotting protocol (Invitrogen), using a 4-12% Novex Bis-Tris gel and PVDF membrane. The membrane was transferred into 0.1% Ponceau S stain (Sigma) to check that the protein had transferred, and it was cut at the level of the 40 kDa protein standard to allow simultaneous detection of the GAD proteins (65-67 kDa) and the EGFP control (29 kDa).

The membrane was blocked by incubating in 5% skim milk powder in TBST on a shaker overnight at 4° C. then was washed three times with TBST (5 minutes per washing) prior to addition of the anti-GAD65/67 antibody (1:2000, AB1511 Chemicon) to the top half of the membrane, and the anti-GFP antibody (1:2000, A290, Abcam) to the bottom half of the membrane. Both antibodies were diluted in 1% milk powder/TBST. Following an overnight incubation, the membranes were washed three times with TBST (5 minutes per washing) then incubated at room temperature for one hour in goat anti-rabbit secondary antibody (1:30,000, Sigma A6158) diluted in 1% milk powder/TBST. The membranes were washed three times with TBST (5 minutes per washing) prior to detection using the ECL Plus detection kit (Amersham Biosciences) according to the manufacturer's instructions. The membrane was exposed against X-ray film (Kodak) for two minutes, then developed.

Protein levels were quantified using Adobe Photoshop. A standard curve of GAD protein (ng) was generated from the GAD standards using Microsoft Excel. The amount of GAD protein produced from transfection of human GAD65, consensus GAD65 and cat GAD65 could then be calculated.

Production of rAAV Vectors

Recombinant AAV vectors were packaged in HEK 293 cells and purified by heparin affinity chromatography (Clark K R, Liu X, McGrath J P, Johnson P R. (1999) Hum Gene Ther. 10, 1031-9).

Infusion of Vectors into the Rat STN

Rats were anesthetized with 1.2 ml/kg Pentobarb 300 diluted 1:5 in sterile PBS and mounted on a stereotaxic frame. A small burr hole was drilled in the skull and 2 μl of vector plus 1 μl of 20% manitol (3 μl total volume) was infused into the left STN at AP −3.8, ML +2.4, DV −8.0, at a rate of 100 nl/min. Anterior-posterior and medial-lateral coordinates are respective to Bregma, and dorsal-ventral coordinates are respective to the skull surface.

### Sacrificing and Immunohistochemistry

Rats were sacrificed with an overdose of Pentobarb 300 and perfused transcardially with 0.9% saline and formalin. Brains were then removed and stored in formalin for an additional 1-2 days before being cryoprotected in 30% sucrose solution. Free-floating 40 μm coronal sections were cut on a Leica freezing microtome. Immunohistochemistry was performed as per standard protocol using anti-GAD (Sigma G5163 1:2000, rabbit secondary) antibodies, and visualized using the DAB method.

### Example 2

### Consensus GAD65

### Plasmid Construction

The consensus GAD65 gene was synthesized by GenScript Corporation (Piscataway, N.J.) with an XhoI and EcoRI linker on the 5′ and 3′ end, respectively. The construct was digested with XhoI and EcoRI and subcloned into the pAAV backbone plasmid pAAV/CBA-pl-WPRE-BGH to form pAAV/CBA-consGAD65-WPRE-BGH.

### Transfection for Protein Quantification

HEK 293 cells were plated at 5×10⁴ cells/well onto collagen-coated plates, 24 hours prior to transfection. For transfection, 1 μg of the appropriate plasmid DNA (pAM/CBA-hGAD65-WPRE-BGH, pAM/CBA-consGAD65-WPRE-BGH, pAM/CBA-hGAD67-WPRE-BGH, pAM/CBA-catGAD65-WPRE-BGH) spiked with 0.1 μg of EGFP plasmid was mixed with 50 μl OptiMEM (Invitrogen) in a sterile tube. When GAD65 and GAD67 were co-transfected, 0.5 μg of each plasmid was used. Three microlitres of Optifect (Invitrogen) was mixed with 50 μl OptiMEM in a separate tube, then the contents of the tubes were mixed and incubated at room temperature for 30 min. The plasmid/Optifect solution was then added to a well and mixed by gently pipetting up and down. The transfection mix was incubated with the cells for 48 hours prior to extraction of cytoplasmic proteins.

### Western Blotting

The amount of protein in each lysate was quantified using the Total Protein Microassay (Biorad) with BSA from 0 to 25 ng/ml as a standard. One and a half micrograms of each sample and 10, 20, 40 and 60 ng samples of recombinant human GAD65 (rhGAD65, Diamyd) were processed for SDS-PAGE and Western Blotting analysis according to the Novex NuPage western blotting protocol (Invitrogen), using a 4-12% Novex Bis-Tris gel and PVDF membrane. The membrane was transferred into 0.1% Ponceau S stain (Sigma) to check that the protein had transferred, and it was cut at the level of the 40 kDa protein standard to allow simultaneous detection of the GAD proteins (65-67 kDa) and the EGFP control (27 kDa).

The membrane was blocked by incubating in 5% skim milk powder in TBST on a shaker overnight at 4° C. then was washed three times with TBST (5 minutes per washing) prior to addition of the anti-GAD65/67 antibody (1:2000, AB1511 Chemicon) to the top half of the membrane, and the anti-GFP antibody (1:2000, A290, Abcam) to the bottom half of the membrane. Both antibodies were diluted in 1% milk powder/TBST. Following an overnight incubation, the membranes were washed three times with TBST (5 minutes per washing) then incubated at room temperature for one hour in goat anti-rabbit secondary antibody (1:30,000, Sigma A6158) diluted in 1% milk powder/TBST. The membranes were washed three times with TBST (5 minutes per washing) prior to detection using the ECL Plus detection kit (Amersham Biosciences) according to the manufacturer's instructions. The membrane was exposed against X-ray film (Kodak) for two minutes, then developed.

Protein levels were quantified using Adobe Photoshop. A standard curve of GAD protein (ng) was generated from the GAD standards using Microsoft Excel. The amount of GAD protein produced from transfection of human GAD65, consensus GAD65 and cat GAD65 could then be calculated.

### Transfection for GABA Release Experiment

HEK293 cells were plated onto a 12-well plate one day before transfection in DMEM supplemented with 10% FBS. Cells in each well were transfected with 500 ng of a plasmid mix consisting of pAAV/CBA-hGAD67-WPRE-BGH (200 ng) and pAAV/CBA-EGFP-WPRE-BGH (100 ng) plus either pAAV/CBA-hGAD65-WPRE-BGH (human GAD65) (200 ng), pAAV/CBA-consGAD65-WPRE-BGH (200 ng). The control cells were transfected with pAAV/CBA-pl-WPRE-BGH (empty vector control) (400 ng) and pAAV/CBA-EGFP-WPRE-BGH (100 ng). The transfections were performed in triplicates using FuGene6 (Roche). After 48 hours the media was removed and the cells were washed twice with PBS.

### GABA Release

To measure secreted GABA, pre-warmed PBS (1 ml) was added and the cells were incubated for 15 min at 37° C. An aliquot (0.4 ml) was then removed and centrifuged at 1,000×g to pellet any floating cells. The samples were immediately frozen at −80° C. GABA HPLC analysis was performed at Neurochemistry Core Laboratory of Vanderbilt University, Nashville, Tenn.

### Example 3

### Vectors and Delivery Systems

This series of prophetic examples describes methods of construction and use of various adeno-associated virus delivery systems for Consensus or Feline GAD65 in addition to the vectors described in the previous examples.

### Vector Construction

This example describes a method for construction of an adeno-associated virus vector with a novel GAD cDNA. The novel GAD of the invention can be subcloned into an AAV plasmid under the control of a 1.8 kb rat NSE (neuron specific enolase) promoter (Foress-petter et al. (1986) J. Neurosci. Res. 16, 141-156 (1998)) 5′ of the GAD cDNA followed by the Woodchuck Hepatitis Post-Transcriptional Regulatory Element (WPRE) and a bovine growth hormone (BGH) polyadenylation site between the AAV inverted terminal repeats, as previously described (During et al. (1998) Nature Med. 4:1131-1135).

Plasmids can be packaged to generate high titer rAAV-Novel GAD viral particles using an optimized protocol based on the original helper-free transient transfection method described by Samulski et al. (1989) J. Virol. 63:3822-3828), but modified by using an improved 4th generation helper plasmid, pDG as described by Grimm et al. (1999) Hum Gene Ther 10, 2445-2450. The helper plasmid would contain both the rep and cap open reading frames, as well the minimal set of adenoviral genes necessary for helper functions. The vectors can generated using calcium phosphate transfection of both plasmids into 293 cells. Vector stocks can be purified using ammonium sulfate followed by double cesium banding. The bands containing the viral particle can then be isolated from the cesium chloride preparation and dialysis into suitable buffer.

Particle titers can be determined using an ELISA assay kit available (Progen, Inc.) which uses an A20 monoclonal antibody that recognizes intact particles. Purification of the viral particles can be performed as described by Clark et al., (1999) Hum. Gene. Ther. 10: 1031-1039 and Zolutkhin et al. (1999) Gene Therapy 9: 973-985.

### Packaging Protocol

To package the recombinant vectors, human embryonic kidney cells, 293 cells (from American Type Culture Collection (ATCC # CRL-1573)), passage 4-12 can be used. The 293 kidney cells (1.5×10⁷ cells) can be seeded into forty 15 cm dishes in complete DMEM (Gibco) containing 10% fetal bovine serum (Hyclone), 0.1 mM MEM non-essential amino acids solution (Gibco), 1 mM MEM sodium pyruvate (Gibco), 0.05% Penicillin-Streptomycin (5,000 units/ml, Gibco), and incubated overnight at 37° C. When the cells are 70% confluent and 2-3 hours prior to transfection, the cells should be fed fresh Iscove modified Dulbecco medium (IMDM, Gibco) containing 10% fetal bovine serum (Hyclone) without antibiotics.

Plasmids can be isolated from the cells by the alkaline lysis method (Sambrook et al., supra), and were further purified by HPLC (BioCAD, Sprint, PerSeptive Biosystems), and concentrated with 2 volumes of 100% ethanol (AR grade, BDH). All HPLC elute buffers (Buffer A: 250 mM Tris HCl, 10 mM EDTA, pH 8.0; Buffer B: 25 mM Tris HCl, 1 mM EDTA, 2M NaCl, pH, 8.0; Buffer C: Milli Q water) used for purification should be autoclaved and filter sterilized prior to use. For each 15 cm tissue culture plate, a total of 60 μg of plasmid DNA should be dissolved in 3.0 ml of 0.25M CaCl₂ and then quickly mixed with 3.0 ml of HEPES-buffered saline (50 mM HEPES, 280 mM NaCl, 1.5 mM Na₂HPO₄ [pH 7.05-7.10]), incubated for 2 min and then added to the cells. 6-8 hours after transfection, the medium is to be aspirated and cells washed with IMDM supplemented with 10% fetal bovine serum without antibiotics. The washing medium is to then aspirated and replaced with fresh IMDM (Gibco) containing 10% fetal bovine serum with trace pen/strep. The cells are to be harvested at 48 hours after transfection. After low-speed centrifugation on a tabletop centrifuge, the cell pellets are resuspended in 20 ml of Opti-MEM (Gibco) and subjected to sonication using 15-20% energy for 50 bursts lasting 1 min. Cell debris is removed with low speed centrifugation. The clarified supernatant is collected into a 50 ml polypropylene tube, the cell pellets are resuspended in 20 ml of Opti-MEM for reextraction. The supernatants are then combined.

One-third volume of ice-cold saturated (NH₄)₂SO₄ is added to the supernatant, mixed and placed on ice for 10 minutes. The sample is then centrifuged at 8,000 rpm at 4° C. for 10 min, supernatant is transferred to a polypropylene centrifuge tube, ⅔ volume of the initial lysate of saturated (NH₄)₂SO₄ is added and mixed well, then placed on ice for 20 min prior to centrifugation at 12,000 rpm for 20 min at 4° C. The pellet is redissolved in CsCl-phosphate-buffered saline (PBS) (pH 7.4) solution (density 1.37 g/l) and centrifuged in an SW41 rotor Beckman at 80,000 rpm (for 24 hours with a 0.5 ml CsCl-PBS cushion (density, 1.5 g/ml).

The band containing recombinant AAV particle (rAAV) is collected and re-centrifuged as described above for a further 24 hours. Finally, the rAAV band is collected following the second CsCl centrifugation and dialyzed against one liter sterile dialysis buffer containing 50 mM NaCl, 5 mM Tris-HCl and 0.5 mM MgCl₂ (pH 7.4) for an initial 4 hours. Dialysis is repeated using one liter of fresh cold sterile dialysis buffer for another 4 hours and finally overnight dialysis using a 50,000 molecular weight cut off dialysis membrane (Spectrapor) and fresh sterile dialysis buffer. The AAV virus particle titer can determined using an ELISA method described by Wistuba et al. ((1997) J. Virol. 71: 1341-1352). Briefly, a monoclonal antibody specific for AAV assembled capsids is coated onto microtiter strips and is used to capture AAV particles. A biotin-conjugated monoclonal antibody to AAV is bound to the immune complex, streptavidin peroxidase conjugate reacts with the biotin molecules. Addition of substrate solution results in a color reaction which is proportional to specifically bound virus particles, and allows the quantitative determination of an unknown particle titer.

Viral particle titre can also determined by the AAV titration ELISA kit provided by Progen (Germany). One hundred microliter of ready-to-use wash buffer, positive, negative controls, and dilutions of standard and samples are pipetted into appropriate wells of the microtiter strips which were sealed with adhesion foil. After incubation for 1 hour at 37° C., the solution is removed and each well is rinsed 3 times with 200 μl of washing buffer for 30 seconds. The washing buffer is removed and 100 μl of ready to use biotin conjugate is added. The strips are sealed with adhesion foil and incubated for one hour at 37° C. The strips are washed as described above. A volume of 100 μl of ready-to-use streptavidin conjugate is added, and the strips are sealed with adhesion foil and incubated for one hour at 37° C. The washing steps are then repeated as described above. Substrate at a volume of 100 μl are pipetted into each well and incubated at room temperature for 10 min. The reaction is stopped by adding 100 μl of stop solution into each well. Absorbance of each well can be measured photometrically at 450 nm wavelength.

### Transduction of Neurons

Target cells for vectors of the invention include, among other, the intrinsic neurons of the subthalamic nucleus (STN). The vector of the invention can be administered at a dose of 3.5×10⁹ virions in a volume of 35 microliters (based on genomic titer of rAAV stocks of 10¹¹/ml) with an additional 15 μl of USP 25% mannitol as a flush. Based on the extensive analysis of vector distribution using AAV in the rodent brain, it has been shown that if rAAV is delivered at low infusion rates (<10 μl/min), the best transduction levels were obtained. Moreover the vector is delivered with high efficiency to cells immediately surrounding the injection tract, with an exponential fall off in gene expression extending from the tip of the injection cannula. Using volumes of 3 microliters delivering ˜5×10⁹ virions, 80% of transduced cells lie within 1 mm of the injection site with less than 5% of transduced cells lying greater than 2 mm from the injection site. In the study using a 35 μl volume of vector (12 fold greater volume) but a titer approximately 15-20 fold lower (i.e. roughly equivalent number of vector genomes delivered), gene expression was restricted to a volume of several millimeters. This would confine the vector to the STN whose dimensions are approximately 4.8 mm×5 mm×6 mm or ˜140 mm. Similar techniques can be used to confine the virion to any specified region.

### Efficiency of Transduction

Transduction efficiencies can reach 100% in permissive cell-lines and permissive target cells in vivo if sufficient MOI are used. Based on rodent data it is expected that an injection volume of 35 microliters into a human STN with the absolute number of virion genomic particles of ˜3.5×10⁹ is likely to transduce from 70-175,000 cells. This represents approximately 25-60% of target cells transduced.

Those skilled in the art will recognize, or be able to ascertain using no more than routine experimentation, many equivalents to the specific embodiments of the invention described herein. Such equivalents are intended to be encompassed by the following claims.

### REFERENCES

Asada et al., 1996. Mice lacking the 65 kDA isoform of glutamic acid decarboxylase (GAD65) maintain normal levels of GAD67 and GABA in their brains but are susceptible to seizures. BBRC 229, 891-895.
Asada et al., 1997. Cleft palate and decreased brain gamma-aminobutyric acid in mice lacking the 67-kDa isoform of glutamic acid decarboxylase. PNAS 94, 6469-6499.
Erlander M G et al., 1991 Two genes encode distinct glutamate decarboxylases. Neuron 7, 91-100.
Bu D-F et al., 1992. Two human glutamate decarboxylases, 65-kDa GAD and 67-kDa GAD are each encoded by a single gene. PNAS 89, 2115-2119.
Dirkx R et al., 1995. Targeting of the 67-kDa isoform of glutamic acid decarboxylase to intracellular organelles is mediated by its interaction with the NH₂-terminal region of the 64-kDa isoform of glutamic acid decarboxylase. J Biol Chem 270, 2241-2246.
Esclapez M et al., 1994. Comparative localization of two forms of glutamic acid decarboxylase and their mRNAs in rat brain supports the concept of functional differences between the forms. J Neurosci 14, 1834-1855.
Kanaani et al., 1999. The hydrophilic isoform of glutamate decarboxylase, GAD67, is targeted to membranes and nerve terminals independent of dimerization with the hydrophobic membrane anchored isoform, GAD65. J Biol Chem 274, 37200-37209.
Kanaani et al., 2002. A combination of three distinct trafficking signals mediates axonal targeting and presynaptic clustering of GAD65. J Cell Biol. 158, 1229-1238.
Kash S F et al., 1997. Epilepsy in mice deficient in the 65-kDa isoform of glutamic acid decarboxylase. PNAS 94, 14060-14065.
Namchuk M et al., 1997. Phosphorylation of serine residues 3, 6, 10 and 13 distinguishes membrane anchored from soluble glutamic acid decarboxylase 65 and is restricted to glutamic acid decarboxylase65alpha. J. Biol Chem 272, 1548-1557.
Sheikh S N and Martin D L. 1996. Heteromers of glutamate decarboxylase isoforms occur in rat cerebellum. J Neurochem 66, 2082-2090.
Stork, O et al., 2000. Postnatal development of a GABA deficit and disturbance of neural functions in mice lacking GAD65. Brain Res 865, 45-58.

What is claimed is:

1. A polypeptide comprising the amino acid sequence of SEQ. ID NO: 2 and exhibiting a glutamic acid decarboxylase activity.

2. A polypeptide comprising the amino acid sequence of SEQ. ID NO: 4 and exhibiting a glutamic acid decarboxylase activity.

3. A polypeptide comprising an amino acid sequence having a 60% or more homology with the amino acid sequence of SEQ. ID NO: 2, and exhibiting a glutamic acid decarboxylase activity.

4. A polypeptide comprising an amino acid sequence having a 60% or more homology with the amino acid sequence of SEQ. ID NO: 4, and exhibiting a glutamic acid decarboxylase activity.

5. A polynucleotide encoding the polypeptide according to claim 1.

6. A polynucleotide encoding the polypeptide according to claim 2.

7. A polynucleotide encoding the polypeptide according to claim 3.

8. A polynucleotide encoding the polypeptide according to claim 4.

9. A vector comprising the polynucleotide according to claim 2.

10. A host cell comprising the polynucleotide according to claim 2.

11. A medicament comprising the polypeptide according to claim 2 or a fragment thereof, the polynucleotide according to claim 6, or the vector according to claim 9.

12. A pharmaceutical composition comprising the polypeptide according to claim 2 or a fragment thereof, the polynucleotide according to claim 6, or the vector according to claim 9, and a pharmaceutically or veterinary acceptable carrier or diluent.

13. An antibody or a fragment thereof, which binds to the polypeptide according to claim 2.

14. An antibody or a fragment thereof, which binds to the polypeptide according to claim 1.

15. The antibody of any one of claims 13-14, wherein the antibody is a monoclonal antibody.

16. An antibody that specifically binds to a polypeptide produced recombinantly by a host cell comprising the polynucleotide consisting of SEQ ID NO: 1.

17. An antibody that specifically binds to a polypeptide produced recombinantly by a host cell comprising the polynucleotide consisting of SEQ ID NO: 3.

18. An antibody that specifically binds to a polypeptide produced recombinantly by a host cell comprising a polynucleotide encoding the amino acid sequence of SEQ ID NO: 2.

19. An antibody that specifically binds to a polypeptide produced recombinantly by a host cell comprising a polynucleotide encoding the amino acid sequence of SEQ ID NO: 4.

20. The antibody of claims 18 or 19, wherein the host cell is a mammalian cell.

21. A method for treating a neurodegenerative disease in a subject comprising:
identifying a target site in the central nervous system that requires modification;
delivering a vector comprising a nucleotide sequence encoding the polypeptide of claim 1 to the target site in the central nervous system; and
expressing the polypeptide of claim 1 the target site to treat or reduce the neurodegenerative disease.

22. A method for treating a neurodegenerative disease in a subject comprising:
identifying a target site in the central nervous system that requires modification;
delivering a vector comprising a nucleotide sequence encoding the polypeptide of claim 2 to the target site in the central nervous system; and
expressing the polypeptide of claim 1 the target site to treat or reduce the neurodegenerative disease.

23. The method of claim 22, wherein the vector is a viral vector.

24. The method of claim 23 wherein the a viral vector is selected from the group consisting of adenovirus vectors, herpes virus vectors, parvovirus vectors, and lentivirus vectors.

25. The method of claim 24, wherein the a viral vector is an adeno-associated viral vector.

26. The method of claim 21, wherein the vector is a non-viral vector.

27. The method of claim 25, wherein the non-viral vector is a liposome-mediated delivery vector.

28. The method of claim 22, wherein the vector is delivered using stereotaxic delivery.

29. The method of claim 22, wherein the target site in the central nervous system is a region of the brain.

30. The method of claim 29 wherein the region of the brain is selected from the group
consisting of basal ganglia, subthalamic nucleus (STN), pedunculopontine nucleus (PPN), substantia nigra (SN), thalamus, hippocampus, amygdala, hypothalamus, cortex, and combinations thereof.

31. The method of claim 29, wherein the region of brain is the hippocampus.

32. The method of claim 29, wherein the region of brain is the amygdala.

33. The method of claim 29, wherein the region of brain is the hypothalamus.

34. The method of claim 22, wherein the neurodegenerative disease is selected from
the group consisting of Parkinson's disease, Alzheimer's disease, senile dementia, Amyloid Lateral Sclerosis (ALS), and epilepsy.

35. A method for treating epilepsy in a subject comprising:
identifying one or more regions of the brain that require modification;
delivering a vector comprising a nucleotide sequence encoding the polypeptide of claim 2 to the identified region of the brain; and
expressing said polypeptide in the region of the brain to treat or reduce epilepsy.

36. The method of claim 35, wherein the vector is a viral vector.

37. The method of claim 36, wherein the a viral vector is selected from the group
consisting of adeno-associated viral, adenovirus vectors, herpes virus vectors, parvovirus vectors, and lentivirus vectors.

38. The method of claim 36, wherein the a viral vector is an adeno-associated viral vector.

39. A method of altering expression of glutamic acid decarboxylase (GAD) in a region of the central nervous system (CNS) of a subject with epilepsy comprising:
identifying a target site in the CNS that requires modification;
delivering a vector comprising a nucleotide sequence encoding the polypeptide of claim 2 to the target site in the CNS; and
expressing the polypeptide of claim 2 in the target site.

40. The method of claim 39, wherein the vector is a viral vector.

41. The method of claim 50, wherein the a viral vector is selected from the group consisting of adenovirus vectors, herpes virus vectors, parvovirus vectors, and lentivirus vectors.

42. The method of claim 39, wherein the a viral vector is an adeno-associated viral vector.

43. The method of claim 39, wherein the vector is a non-viral vector.

44. The method of claim 43, wherein the non-viral vector is a liposome-mediated delivery vector.

45. The method of claim 39, wherein the vector is delivered using stereotaxic delivery.

46. The method of claim 39, wherein the target site in the central nervous system is a region of the brain.

47. The method of claim 46, wherein the region of the brain is selected from the group consisting of basal ganglia, subthalmic nucleus (STN), pedunculopontine nucleus (PPN), substantia nigra (SN), thalmus, hippocampus, amygdala, hypothalamus cortex, and combinations thereof.

48. The method of claim 47, wherein the region of brain is the hippocampus.

49. The method of claim 47, wherein the region of brain is the amygdala.

50. The method of claim 47, wherein the region of brain is the hypothalamus.

51. A vector for expression of the polypeptide of claim 2 in cells of the central nervous system comprising:
a tissue specific promoter operably linked to a nucleotide sequence encoding GAD; and
a post-transcriptional regulatory element.

52. The vector of claim 51, wherein the vector is selected from the group consisting of adeno-associated vector, adenovirus vectors, herpes virus vectors, parvovirus vectors, and lentivirus vectors.

53. The vector of claim 52, wherein the vector is an adeno-associated vector.


SEQUENCE LISTING (11 sequences)

SEQ ID NO: 1 - DNA, 1766 bp, Felis catus
atggcatctccgggctctggcttttggtctttcgggtcggaagatggctctggggatcccgagaatcccggcacagcgcgagcctggtgccaagtggctcagaagttcacgggcggcatcggaaacaaactgtgcgccctgctctacggagacgccgagaagccggcggagagcggcgggagccaacccccgcgggccacctcccggaaggccgcctgcgcctgcaaccagaagccctgcagctgccccaaagccgatgtcaactacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgctgatcgcacgctttaagatgttcccagaagtcaaggagaaaggaatggctgctgtgcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacacaagatgatgggagtccctttgcagtgctctgctctcctggttagagaagagggattgatgcagagctgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatatcgataaatgtttggagttggcagagtatttatacaacatcataaaaaaccgagaaggatatgagatggtgtttgatgggaagcctcagcacacaaatgtctgcttctggtacgtgcctccaagcttgcgtgtgctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagatttataataaatatt

SEQ ID NO: 2 - protein, 585 aa, Felis catus
MASPGSGFWSFGSEDGSGDPENPGTARAWCQVAQKFTGGIGNKLCALLYGDSEKPAESGGSQPARATSRKATCACNQKPCSCPKADVNYAFLHATDLLPACDGERPTLAFLQDVMGILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMLIARFKMFPEVKEKGMAAVPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQSCNQMHASYLFQQDKHYDLSYDTGDKALQCGRHVDVFKLWLMWRAKGTTGFEAHIDKCLELAEYLYNIIKNREGYEMVFDGKPQHTNVCFWYVPPSLRVLEDNEERMSRLSKVAPVIKARMMEYGTTMVSYQPLGDKVNFFRMVISNPAATHQDIDFLIEEIERLGQDL

SEQ ID NO: 3 - DNA, 1766 bp, Unknown; identical to SEQ ID NO: 1
  consensus protein sequence from felis domesticus, Canis lupus familiaris, Mus musculus, Bos taurus, Sus scrofa, Macaca mulatta, Rattus norvegicus and Homo sapiens

SEQ ID NO: 4 - protein, 585 aa, Unknown
  consensus protein sequence from felis catus, Canis lupus familiaris, Mus musculus, Bos taurus, Sus scrofa, Macaca mulatta, Rattus norvegicus and Homo sapiens
MASPGSGFWSFGSEDGSGDPENPGTARAWCQVAQKFTGGIGNKLCALLYGDAEKPAESGGSQPPRATSRKAACACNQKPCSCPKADVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMLIARFKMFPEVKEKGMAAVPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQSCNQMHASYLFQQDKHYDLSYDTGDKALQCGRHVDVFKLWLMWRAKGTTGFEAHIDKCLELAEYLYNIIKNREGYEMVFDGKPQHTNVCFWYVPPSLRVLEDNEERMSRLSKVAPVIKARMMEYGTTMVSYQPLGDKVNFFRMVISNPAATHQDIDFLIEEIERLGQDL

SEQ ID NO: 5 - protein, 585 aa, Canis lupus
MASPGSGFWSFGSEDGSGDPENPSTARAWCQVAQKFTGGIGNKLCALLYGDAEKPAESGGSEPPRATSRKAACACNQKPCSCPKAEVNYAFLHATDLLPACDGERPTLAFLQDVMDILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMLIARFKMFPEVKEKGMAAVPRLIAFTSEHSHFSLKKGAAALGIGTDSVILIKCDERGKMVPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQSCNQMHASYLFQQDKHYDLSYDTGDKALQCGRHVDVFKLWLMWRAKGTTGFEAHIDKCLELAEYLYSIIKNREGYEMVFDGKPQHTNVCFWYVPPSLRVLEDNEERMNRLSKVAPVIKARMMEYGTTMVSYQPLGDKVNFFRMVISNPAATHQDIDFLIEEIERLGQDL

SEQ ID NO: 6 - protein, 585 aa, Mus musculus
MASPGSGFWSFGSEDGSADPENPGTARAWCQVAQKFTGGIGNKLCALLYGDSGKPAEGGGSVTSRAATGKVACTCDQKPCNCPKGDVNYAFLHATDLLPACDGERPTLAFLQDVMNILLQYVVKSFDRSTKVIDFHYPNELLQEYNWELADQPQNLEEILTHCQTTLKYAIKTGHPRYFNQLSTGLDMVGLAADWLTSTANTNMFTYEIAPVFVLLEYVTLKKMREIIGWPGGSGDGIFSPGGAISNMYAMLIARYKMFPEVKEKGMAAVPRLIAFTSEHSHFSLKKGAAALGIGTDSVILIKCDERGKMIPSDLERRILEVKQKGFVPFLVSATAGTTVYGAFDPLLAVADICKKYKIWMHVDAAWGGGLLMSRKHKWKLSGVERANSVTWNPHKMMGVPLQCSALLVREEGLMQSCNQMHASYLFQQDKHYDLSYDTGDKALQCGRHVDVFKLWLMWRAKGTTGFEAHIDKCLELAEYLYTIIKNREGYEMVFDGKPQHTNVCFWFVPPSLRTLEDNEERMSRLSKVAPVIKARMMEYGTTMVSYQPLGDKVNFFRMVISNPAATHQDIDFLIEEIERLGQDL

SEQ ID NO: 7 - protein, 585 aa, Bos taurus
MASPGSGFWSFGSEDGSGDPENPGTARAWCQVAQKFTGGIGNKLCALLYGDAEKPAESSGSQPPRATSRKAACSCNQKPCSCPKAEVNYAFLHATDLLPACDGERPTLAFLQEVMDILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMLIARFKMFPEVKEKGMAAVPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQSCNQMHASYLFQQDKHYDLSYDTGDKALQCGRHVDVFKLWLMWRAKGTIGFEAHIDKCLELAEYLYNIIKNREGYEMVFDGKPQHTNVCFWYVPPSLRVLDNNEERLSRLSKVAPMIKARMMESGTTMVSYQPLGDKVNFFRMVISNPAATHQDIDFLIEEIERLGQDL

SEQ ID NO: 8 - protein, 585 aa, Sus scrofa
  misc_feature (81)..(81): Xaa can be any naturally occurring amino acid
MASPGSGFWSFGSEDGSGDPENSGTARAWCQVAQKFTGGIGNKLCALLYGDAEKPAESGGSQPPRTTSRKATCACNQKPCXCPKAEVNYAFLHATDLLPACDGERPTLAFLQDVMDILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMLIARFKMFPEVKEKGMAAVPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQSCNQMHASYLFQQDKHYDLSYDTGDKALQCGRHVDVFKLWLMWRAKGTTGFEAHIDKCLELAEYLYNIIKNREGYEMVFDGKPQHTNVCFWYVPPSLRVLDNNEERMSRLSKVAPVIKARMMESGTTMVSYQPLGDKVNFFRMVISNPAATHQDIDFLIEEIERLGQDL

SEQ ID NO: 9 - protein, 540 aa, Macaca mulatta
MASPGSGFWSFGSENGSGDSDNPGTARAWCQVAQKFTGGIGNKLCALLYGDAEKPAESGGSQPPRVAARKAACACDQKPCSCPKADVNYGFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAAVPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSPLSPLTICPPHRL

SEQ ID NO: 10 - protein, 585 aa, Rattus norvegicus
MASPGSGFWSFGSEDGSGDPENPGTARAWCQVAQKFTGGIGNKLCALLYGDSEKPAESGGSVTSRAATRKVACTCDQKPCSCPKGDVNYALLHATDLLPACEGERPTLAFLQDVMNILLQYVVKSFDRSTKVIDFHYPNELLQEYNWELADQPQNLEEILTHCQTTLKYAIKTGHPRYFNQLSTGLDMVGLAADWLTSTANTNMFTYEIAPVFVLLEYVTLKKMREIIGWPGGSGDGIFSPGGAISNMYAMLIARYKMFPEVKEKGMAAVPRLIAFTSEHSHFSLKKGAAALGIGTDSVILIKCDERGKMIPSDLERRILEVKQKGFVPFLVSATAGTTVYGAFDPLLAVADICKKYKIWMHVDAAWGGGLLMSRKHKWKLNGVERANSVTWNPHKMMGVPLQCSALLVREEGLMQSCNQMHASYLFQQDKHYDLSYDTGDKALQCGRHVDVFKLWLMWRAKGTTGFEAHIDKCLELAEYLYNIIKNREGYEMVFDGKPQHTNVCFWFVPPSLRVLEDNEERMSRLSKVAPVIKARMMEYGTTMVSYQPLGDKVNFFRMVISNPAATHQDIDFLIEEIERLGQDL

SEQ ID NO: 11 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL